(19) United States
(12) Patent Application Publication
(10) Pub. No.: US 2011/0313536 A1
(43) Pub. Date: Dec. 22, 2011

(54) FUNCTIONALIZED TITANIUM IMPLANTS AND RELATED REGENERATIVE MATERIALS

(75) Inventor: Takahiro Ogawa, Torrance, CA (US)

(73) Assignee: The Regents of the University of California, Oakland, CA (US)

(21) Appl. No.: 13/131,013

(22) PCT Filed: Nov. 24, 2009
(86) PCT No.: PCT/US09/65816

§ 371 Date: Sep. 6, 2011

Related U.S. Application Data
(62) Division of application No. 61/117,831, filed on Nov. 25, 2008.

(51) Int. Cl. A61F 2/28; B01J 19/12

(52) U.S. Cl. 623/23.53; 204/157.4

(57) ABSTRACT

It is provided a method for functionalizing an implant comprising treating the implant surface thereby causing the surface to be electro-positively charged. The implant has enhanced tissue-implant integration and/or bone-implant integration.

### CROSS REFERENCE

This application claims priority to U.S. Provisional Application No. 61/117,831 filed on Nov. 25, 2008, the teaching of which is incorporated by reference in its entirety.

### FIELD OF THE INVENTION

This invention generally relates to a medical implant for biomedical uses.

### BACKGROUND

Osteoporotic femoral neck fracture and degenerative changes of knee and hip joints are quite common problem. Over 500,000 procedures are performed annually in the United States for hip and knee reconstruction in which the use of titanium implants as anchor has become an essential treatment modality. The nature and location of bone fracture at these areas do not allow for immobilization of the bone (e.g., cast splinting), and usually immediately after the surgery the implants are impacted by constant and/or cyclic loading caused by gravity and daily life activities such as walking. Issues of such treatment outcome largely include a considerable degree of disability, long-lasting dependence, mortality, relatively high percentage of the revision surgery ranging 5%-40%, and substantial reduction of quality of life. Another detrimental factor is that the implant placement for these purposes faces impaired bone regenerative potential and metabolic activity such as osteoporotic and aged properties which hinder bone healing around implants. Therefore, rapid and firm establishment of bone and joint anchorage using endosseous implants is an ever going effort to minimize the morbidity and maximize functional recovery and long-term prognosis.

Meanwhile, restorative treatment of missing teeth using dental titanium implants is commonly accepted. However, the application of implant therapy in dentistry has various risk factors, including the quality and dimensions of host bone, systemic conditions and age. More importantly, a protracted healing time (4-6 months) required for titanium implants to integrate with bone to endure occlusal load practically limits the application of this beneficial treatment. Implants with improved bone-forming (osteoconductive) capacity would provide considerable benefits to patients and dentists.

In addition to the bone, current tissue regenerative therapies other than bone, joint, and tooth reconstruction therapies encounter many challenges. For instance, currently performed treatments for bone defects after injury and degenerative changes require the use of biological molecules such as growth factors to stimulate the tissue regeneration. And there is still limitations in the effectiveness of the biological molecules and volumes of bone that can be regenerated. Adverse effects of the biological molecules and costs for the treatments are also significant.

Implants with enhanced bioactivity when delivered with carrier biomaterials may have a potential to be used to enhance the biological reaction required for tissue generation.

### SUMMARY

Provided herein is a medical implant which comprises a metallic surface, wherein the metallic surface comprises a metal oxide bearing an electro-positive charge. The metal can be titanium, gold, platinum, tantalum, niobium, nickel, iron, chromium, cobalt, zirconium, aluminum, and palladium. In one embodiment, the implant comprises a carrier material which can be metallic or non-metallic.

In one embodiment, the medical implant comprises a titanium surface. The titanium surface comprises TiO₂. In one embodiment, the titanium surface is substantially free of hydrocarbon.

The implant surface can attract proteins and/or cells at an enhanced rate. The protein can be bovine serum albumin, fraction V, and bovine plasma fibronectin. The cell can be human mesenchymal stem cell and osteoblastic cell. The proteins or cells can attach to the treated implant surface directly, e.g. without a bridging divalent cation.

The implant surface can cause or improve tissue-implant integration and/or bone-implant integration. The implant surface is capable of any of or any combination of the following: increasing adsorption of protein, increasing osteoblast migration, increasing attachment of osteoblasts, facilitating osteoblast spread, increasing proliferation of osteoblast, and promoting osteoblastic differentiation.

Provided herein is a method for functionalizing a medical implants, comprising (1) providing a metallic implant surface, and (2) treating the implant surface thereby causing the surface to be electro-positively charged or enhancing the surface's electro-positive charge. In some embodiments, the method causes the surface to be electro-positively charged in a physiological condition. The physiological condition can have pH value of about 7. In some embodiments, the method causes the surface to be electro-positively charged at a pH lower than 7 or at a pH higher than 7.

In one embodiment, the treated surface is capable of attracting proteins and/or cells at an enhanced rate over untreated surfaces.

In one embodiment, the implant has a titanium surface. In one embodiment, the titanium surface comprises titanium dioxide.

In one embodiment, the implant surface is treated by applying ultraviolet (UV) light to it. The UV light can be applied by a UV lamp. The UV light can be of a wave-length of about 10 nm to 400 nm. In some embodiments, the UV light can be of wavelength of about 170 nm to about 270 nm or about 340 nm to about 380 nm. In some embodiments, the surface is treated by applying a combination of a UV light of a wave-length of about 170 nm to about 270 nm and a UV light of wave-length of about 340 nm to about 380 nm.

The UV light intensity can have a wide range. For example the UV light intensity can be in the range between 0.001 mW/cm² and 100 mW/cm². In some embodiments, the UV light can be of an intensity of about 0.1 mW/cm² or about 2 mW/cm². The treatment with UV light can be over a period of time up to 48 hours, e.g. 30 seconds, 1 minute, 5 minutes, 15 minutes, 30 minutes, 1 hour, 5 hours, 10 hours, 24 hours, 36 hours, and 48 hours.

In one embodiment, the method further comprises processing the implant surface prior to treating the implant surface. The implant surface can be processed by a physical process or a chemical process. The physical process can be machining or sandblasting. The chemical process can be etching by acid or base. The acid can be sulfuric acid. The processed surface can be electro-positively charged. The UV treatment enhances the processed surface's electro-positiveness.

In some embodiments, the treated surface comprises a metal oxide cation. The metal oxide cation can be a titanium oxide cation.

In one embodiment, the treated implant surface can attract a protein such as bovine serum albumin, fraction V, bovine plasma fibronectin. In one embodiment, the treated implant surface can attract a cell such as human mesenchymal stem cell and osteoblastic cell. The proteins or cells can attach to the treated implant surface directly, e.g. without a bridging divalent cation. In one embodiment, the treated titanium surface does not comprise a divalent cation such as Ca²⁺, Mg²⁺, Zn²⁺, etc.

The treated implant surface can enhance tissue-implant integration and/or bone-implant integration at the implant site. The treated implant surface has improved bone-forming capacity over the non-treated implant surface. The treated implant surface is capable of any of or any combination of the following: increasing adsorption of protein, increasing osteoblast migration, increasing attachment of osteoblasts, facilitating osteoblast spread, increasing proliferation of osteoblast, and promoting osteoblastic differentiation.

The above described method can be used for increasing bone forming activity of the implant, increasing osteoconductive capacity of the implant, and enhancing tissue-implant and/or bone-implant integration.

Provided herein is a medical implant which comprises a surface which is functionalized according to the method described above.

### DESCRIPTION OF DRAWINGS

FIG. 1 shows initial bioactivity of acid-etched titanium surfaces with different ages and with or without ultraviolet (UV) treatment.

A Mean±SD adsorption rates of bovine serum albumin after 2, 24 and 72 hours of incubation for newly processed titanium disks used immediately, disks aged for 4 weeks (stored under dark ambient conditions), and disks aged for 4 week and treated with UV.

B Quantity of human mesenchymal stem cells (MSCs) migrated to differently conditioned acid-etched titanium disks through 8-μm holes during 3 hour of incubation.

C Human MSCs attached to the titanium disks evaluated by WST-1 detection 3 and 24 hours after seeding. Data are shown as the mean±SD for all panels (n=3).

FIG. 2 shows initial spread and cytoskeletal arrangement of human mesenchymal stem cells (MSCs) 3 hour after seeding onto differently conditioned acid-etched Ti surfaces: newly processed surface, 4-week-old surface, and UV light-treated 4-week-old surface.

A Representative confocal microscopic images of the cells with staining of rhodamine phalloidin for actin filaments (red), anti-paxillin (green), or a combination of both.

B Cytomorphometric evaluations performed using these images. Data are mean±SD (n=10).

FIG. 3 shows enhanced bone-titanium integration for newly processed and UV-treated acid-etched titanium surfaces compared to the 4-week-old surface, evaluated by biomechanical push-in test. Push-in value of the machined and acid-etched implants with and without light treatment. Data are shown as the mean±SD (n=5).

FIG. 4 shows enhanced albumin adsorption A and cell attachment B to positively charged titanium surfaces.

A Albumin adsorption during 3-hour incubation to various titanium surfaces (newly processed, 4-week-old, and UV-treated 4-week-old surfaces) with and without ion treatment for 24 hours before albumin incubation. The medium for albumin incubation was adjusted at pH 7 or 3.

B The quantity of human MSCs attached to various titanium surfaces during 24 h of incubation. The titanium surfaces were prepared and treated in a same manner as panel A. The culture medium was adjusted at pH 7. Data are shown as the mean±SD (n=3).

FIG. 5 shows a simplified diagram depicting a newly-found electrostatic nature-regulated protein and cellular attachment to titanium surfaces. The left side (Old Titanium) and right side (New or UV-treated Titanium) exhibit cell-phobic and cell-philic surfaces.

FIG. 6 shows generalization of enhanced bioactivity of newly processed and UV-treated titanium surfaces.

A Albumin adsorption during 6-hour incubation to the newly processed, 4-week-old, and UV-treated 4-week-old surfaces of machined titanium and sandblasted titanium disks. Data are shown as the mean±SD (n=3).

B Fibronectin adsorption during 6-hour incubation to the newly processed, 4-week-old, and UV-treated 4-week-old surfaces of machined titanium, acid-etched and sandblasted titanium disks. Data are shown as the mean±SD (n=3).

C Bone-titanium integration measured by the push-in test for the machined implants with and without UV-treatment. Data are shown as the mean±SD (n=5).

FIG. 7 shows Ultraviolet (UV) light-induced osteoblast-affinity titanium surfaces. Two different surface topographies of titanium, machined and acid-etched surfaces, were prepared.

A Superhydrophilic titanium surface obtained after UV light treatment for 48 hours (left images). Changes in hydrophilicity are evaluated by contact angle of H₂O after UV light treatment for various periods of time (line graph).

B Degradation of superhydrophilic status of the titanium surfaces in the dark after 48-hour UV illumination was stopped.

C, D Rates of protein adsorption to the titanium surfaces with and without UV pretreatment. Albumin (C) and fibronectin (D) were incubated on the titanium surfaces for 2, 6, and 24 hours.

E Relative number of osteoblasts attached to titanium surfaces with and without UV pretreatment after 3 and 24 hour incubation, evaluated by WST-I colorimetry.

F, G UV treatment time-dependent changes in titanium affinity to protein and osteoblasts. Albumin adsorption (F) and osteoblast attachment (G) rates of titanium surfaces plotted in association with the hours of UV pre-treatment. Data are shown as the mean±SD (n=3) for panels C-G, and are statistically significant between UV light-treated and untreated control surfaces **p<0.01, *p<0.05, respectively.

FIG. 8 shows initial behavior of osteoblasts on UV-treated titanium.

A Initial osteoblast spread and cytoskeletal arrangement on titanium surfaces with and without UV pretreatment. Confocal microscopic images of osteoblasts 3 hours after seeding with dual staining of DAPI for nuclei (blue) and rhodamine phalloidin for actin filaments (red) (top panels) were taken. Bar is 10 μm. Cell morphometric evaluations were performed using the images (histograms at bottom). Data are shown as the mean±SD (n=6), and are statistically significant between UV light-treated and untreated control surfaces *p<0.05.

B Osteoblast cell density at culture days 2 and 5 on titanium surfaces with and without UV treatment (lower histograms). The fluorescent images of the cells obtained at day 2 are shown on the top to confirm the cell density results.

C Cell proliferative activity of osteoblasts on titanium substrates evaluated by BrdU incorporation per cell at day 2 of culture. Data are shown as the mean±SD (n=3) for panels B and C, and are statistically significant between UV light-treated and untreated control surfaces **p<0.01, *p<0.05, respectively.

FIG. 9 shows enhanced osteoblastic phenotypes and promoted differentiation on UV light-treated titanium surfaces.

A UV-enhanced alkaline phosphatase (ALP) activity, an early-stage maker of osteoblasts. Top panels show images of ALP staining of osteoblastic cells cultured on titanium substrates for 10 days. The ALP-positive area as a percentage of culture area is shown (lower left histogram). Colorimetrically quantified ALP activity standardized per cell is also presented (lower right histogram).

B Mineralizing capability (late-stage marker) of osteoblasts. Top panels show the images of von Kossa mineralized nodule staining of the osteoblasts cultured for 14 days. The Von Kossa positive area as a percentage of culture area is shown (lower left histogram). Total calcium deposition, measured using a colorimetry-based method, is also shown (lower right histogram).

C, D, Expression of bone-related genes in osteoblastic cultures on the machined (C) and acid-etched (D) titanium surfaces. Osteoblasts were cultured on titanium with or without UV light treatment, and gene expression was semi-quantitatively assessed using reverse transcriptase-polymerase chain reaction (RT-PCR). Representative electrophoresis images are shown on top. The quantified level of gene expression relative to the level of GAPDH mRNA expression is presented at the bottom. C: untreated control. UV: UV light-treated. Date are shown as the mean±SD (n=3) for panels A-D, and are statistically significant between UV light-treated and untreated control surfaces **p<0.01, *p<0.05, respectively.

FIG. 10 shows UV light-enhanced bone-titanium integration evaluated by biomechanical push-in test. Push-in value of the machined and acid-etched implants with and without light treatment. Data are shown as the mean±SD (n=5). There is statistical significance between the untreated control and UV light-treated surfaces, **p<0.01; *p<0.05.

FIG. 11 shows UV light-promoted peri-implant bone generation. Representative histological images of the acid-etched titanium implants with Goldner's trichrome stain in an original magnification of ×40 for panels A-D, ×200 for panels E-H, and ×400 for panels I-L are presented. Note that week 2 UV-treated implant is associated with vigorous bone formation that prevents soft tissue from intervening between the bone and implants (arrow heads in F), leading to direct bone deposition onto the implant surface (arrow heads in J). In contrast, the bone around the untreated control appears to be fragmentary (E) and involves soft tissue that migrates into between the bone and implant surface, interfering with the establishment of direct bone-implant contact (arrow heads in I). Such differences in the implant interfacial bone morphogenesis are also clearly seen in the week 4 sections (panels G, H). Extensive bone spread along the implant surface without soft tissue interposition (arrow heads in panel L) is indicated around UV-treated implants (H, L), whereas the bone around the untreated implants is largely kept apart from the implant surface by soft tissue (G and arrow heads in panel K). Average histomorphometric values of bone-implant contact (M), bone volume in the proximal zone (N), bone volume in the distant zone (O), and soft tissue intervention (P) are shown (n=4). Results are statistically significant between the UV light-treated and untreated control surfaces, **p<0.01, *p<0.05, respectively.

FIG. 12 shows UV-light-induced changes in surface characteristics of titanium in association with their biological effects.

A X-ray diffraction (XRD) spectrum of the machined and acid-etched titanium surfaces, as well as TiO₂ pure rutile structure, and a combined structure of rutile and anatase generated by heating at 923K and 673K, respectively.

B Light absorbance spectra of the machined and acid-etched titanium surfaces.

C X-ray photoelectron spectroscopy (XPS) spectrum for the machined and acid-etched titanium surfaces.

D A close-up view of the XPS Ti2p peaks in panel C.

E-G Changes in XPS profile for Ti2p (E), O1s (F) and C1s (G) of the acid-etched titanium surface after various exposure time to UV.

H Changes of atomic percentage of the acid-etched titanium surface with different time periods of UV treatment.

I Plot of albumin adsorption rate after 3-hour incubation against the atomic percentage of carbon on the acid-etched titanium surface, showing a significant inverted linear correlation.

J Osteoblast attachment rate after 3 hour incubation plotted against the atomic percentage of carbon on the acid-etched titanium surface, showing their significant inverted exponential correlation.

K, L Albumin adsorption rate (K) and osteoblast attachment rate (L) plotted against the H₂O contact angle on the acid-etched titanium surface, showing no significant correlation between them.

M Schematic description of a proposed photofunctionalization of TiO₂ illustrating the photogeneration of bio-affinity TiO₂ surface that accelerates and enhances protein adsorption, and attachment and spread of osteoblasts.

FIG. 13 shows the number of cells attached to titanium surface variously treated with UV light.

### DETAILED DESCRIPTION

### Medical Implants Having Metallic Surface

Provided herein is a medical implant which comprises a metallic surface, wherein the metallic surface comprises a metal oxide bearing a positive charge. The metal can be titanium, gold, platinum, tantalum, niobium, nickel, iron, chromium, cobalt, zirconium, aluminum, and palladium. In some embodiments, the metallic surface comprises a metal oxide cation.

### Titanium Surface

Titanium surfaces have been thought to be negatively charged and therefore cations, such as Ca²⁺, react with titanium surfaces. Meanwhile, most proteins and biological cells are negatively charged under physiologic conditions which may be repelled by titanium surfaces.

Titanium implants are used as a reconstructive anchor in orthopedic and dental diseases and problems. Successful implant anchorage depends upon the magnitude of bone directly deposited onto the titanium surface without soft/connective tissue intervention. This is termed “bone-implant integration” or “osseointegration.” Current dental and orthopedic titanium implants have been developed based on this concept and are called “osseointegrated implants.” However, total implant area covered by bone (bone-titanium contact percentage) remains 45±16%, or 50−75%, that is far below the ideal 100%. Most implants fail because of an incomplete establishment or early or late destructive changes of bone-implant interface. The reason that bone tissue does not form entirely around the implant is unknown.

Ultraviolet (UV) light-induced superhydrophilicity of titanium dioxide (TiO₂) was discovered in 1997. The photochemical reaction of semiconductor oxides (including the generation of superhydrophilicity) has earned considerable and broad interest in environmental and clean-energy sciences. The light-generation of a highly hydrophilic titanium surface is ascribed to the altered surface structure of the hydrophilic phase produced by the light treatment. In this model, light treatment creates surface oxygen vacancies at bridging sites resulting in conversion of relevant Ti⁴⁺ sites to Ti³⁺ sites which are favorable for dissociative water adsorption.

The inventor has discovered that 1) newly processed or fabricated titanium surfaces are positively charged; 2) the treatment of old titanium surfaces with UV light makes the surfaces electro-positively charged and the treatment of newly processed titanium surfaces enhances their electropositiveness; 3) these positively charged surfaces are protein- and cell-philic and exhibit substantially increased protein and cell attraction characteristics compared with old titanium surfaces without UV treatment; 4) this newly found and created mechanism of protein and cell attachment enables a direct interaction between proteins and/or cells and titanium surfaces and does not require bridging divalent cations, such as Ca²⁺. The new surface and biological mechanism can be distinguished from the biological process that has been recognized in the field of titanium implants. Because of the enhanced protein adsorption and cell attachment, the resulting titanium surfaces have been demonstrated to exhibit substantially increased tissue integration and regeneration capabilities.

UV-treatment can be performed under a normal ambient air condition, without any atmosphere set-up, such as vacuum or adding inert gas. It is postulated that UV treatment of titanium or titanium-containing metals results in the excitement of electrons from valence band to conduction band of titanium atoms, which results in the creation of positive hole in the superficial layer of titanium and generate the electropositive charge on its surface. To make this electron excitement happen, UV light energy of 3.2 eV is needed, which corresponds to approximately 365 nm wavelength referred to as UVA. In contrast, direct hydrocarbon decomposition is enforced by UVC at its peak wavelength of lower than 260 nm. This carbon removal facilitates the penetration of UVA to the titanium surface and increases the efficiency of the generation of electropositiveness, and eventually expedites and enhances the exposure of the generated electropositive charge.

Without being bound to any theories, a combination of UVA (about 340 nm to about 380 nm) and UVC (about 170 nm to about 270 nm) was used.

UV-treated titanium-mediated enhancement of bone-titanium integration proved to be substantial. For instance, the biomechanical anchorage of acid-etched implants increased up to more than threefold at the early stage of healing at week 2. This threefold increase of the push-in value was obtained at week 8 of healing in the same animal model. In other words, the push-in value obtained by the UV-treated acid-etched implants at week 2 was equivalent to that obtained by untreated acid-etched implants at week 8, indicating that the UV-treated surface accomplished bone-titanium integration 4 times faster. UV-enhanced titanium enabled the optimal level (virtually 100%) of establishment in direct bone-titanium contact with nearly no interposition by soft tissue. These in vivo accomplishments may be due to the following biological processes on UV-treated titanium surfaces: (1) increased adsorption of protein, (2) increased osteoblast migration, (3) increased attachment of osteoblasts, (4) facilitated osteoblast spread, (5) increased proliferation of osteoblasts, and (6) promoted osteoblastic differentiation.

These processes may or may not be independent from each other. For instance, increased protein adsorption may have promoted osteoblastic attachment via enhanced interaction between proteins and cellular integrins. Increased osteoblastic proliferation may have caused the promoted differentiation due to the increased cell-to-cell interaction.

Since the UV-treated surface increased fibronectin adsorption, other cells with RGD-binding integrins may also be attracted to the surface. Interestingly, the intervention of soft tissue was substantially reduced around the UV-treated titanium.

To generate more bone faster, the inverted correlation between proliferation and differentiation rates in osteoblasts must be overcome. This applies to the bone formation around titanium implants. For instance, micro-roughened titanium surfaces have advantages over machined, smooth surfaces in that they not only increase tissue-titanium mechanical interlocking but also promote osteoblastic differentiation, resulting in faster bone formation. The bone mass, however, is smaller than that around the machined surface, in accordance with the diminished osteoblastic proliferation. Acid-etched rougher surface reduces cell density and proliferation activity compared with the relatively smooth machined surface. Rougher surfaces of material substrates generally reduce cell proliferation, where the intracellular tension may be associated with the delay or even restriction of the progression of the G1 phase of the cell cycle. The facilitated spread of the cell on UV-treated surfaces may be an index of relieved intracellular tension. The cell proliferation was evaluated only by BrdU incorporation assay which targets the S phase of the cell cycle.

Analysis to differentiate cells in various cell cycle phases as well as their shape and intracellular tension helps to identify the role of UV-treated surface in regulating osteoblast proliferation. It is found that the rate of osteoblast proliferation increased and that the rate of osteoblastic differentiation as shown in the results of ALP activity and gene expression is slightly elevated. This indicates that UV-treated surfaces enable increasing osteoblastic proliferation without sacrificing differentiation. This biological advantage was well manifested in the histomorphometric result showing the approximately 2-fold increased bone volume around the UV-treated surface.

The UV-mediated enhancement of cellular attachment and proliferation as well as bone-implant contact percentage was demonstrated on deposited titanium tetraisoperoxide with heat treatment to create anatase TiO₂ crystals. The present invention revealed that photo-induced biological effects can be obtained even on the surfaces of titanium bulks without depositing oxidative titanium or sintering.

Another notable finding is that the bone-implant contact obtained in the present invention increased more remarkably than that using the anatase TiO₂ crystals where a bone-implant contact of 28% for 24-hour UV-treated implants and 17% for non-treated implants are reported. The 48-hour UV treatment increased the bone-implant contact 2.5 times at the early healing stage of week 2 in the present study. The intensity, wavelength, and duration of UV light treatment as well as differences in surface chemistry of titanium used have impact on the different biological effects. Prior to the in vivo studies, it is confirmed that 48-hour treatment of UV light was required to generate superhydrophilicity on both machined and acid-etched surfaces and that biological effects, e.g., cell attachment capacity, was on the increase between 24- and 48-hour UV treatment periods.

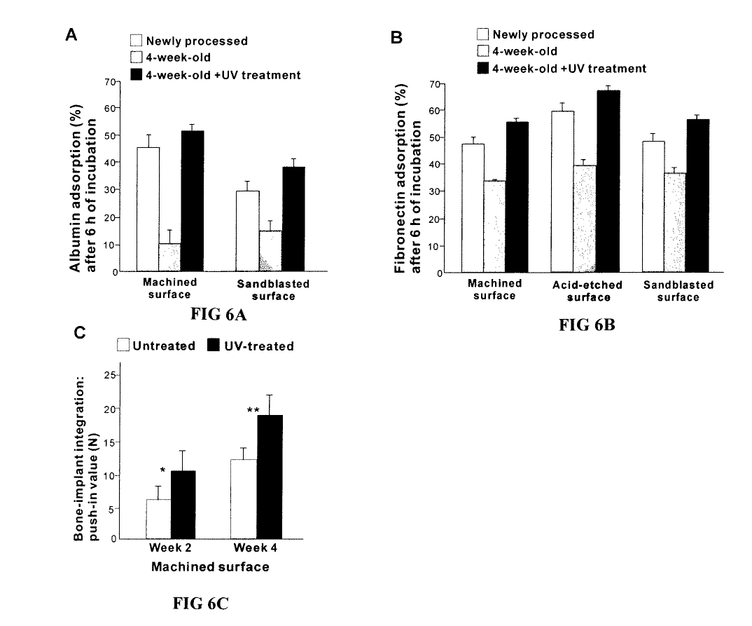

The photogenerated biological effects, as represented by accelerated and enhanced protein adsorption and cell attachment, were associated with generation of superhydrophilicity and decreased percentage of atomic carbon. To determine whether these physicochemical changes are ascribed to photocatalytic phenomena of TiO₂, the titanium surfaces used in this study was carefully characterized. Absorption band at 300-350 nm was found on titanium samples used, which is typically seen on TiO₂ semiconductor. The XPS spectrum revealed a 2p_{3/2} peak at approximately 458.5 eV, but not at 453.8 eV for both machined and acid-etched surfaces (FIG. 6D); the 2P_{3/2} peaks of Ti and TiO₂ are known to be at 453.8 eV and 458.5 eV, respectively. In addition, the shoulder peaks attributed to reduced species such as Ti³⁺ and/or Ti⁰ were not observed in the lower-energy regions for either titanium disks. These data indicated that the near surfaces of these substrates were fully oxidized to form stoichiometric TiO₂ thin layers and that the reduced percentage of carbon with an increase of UV dose was due to the photocatalytic removal of hydrocarbons. Moreover, the data showing that 2p_{3/2} peak was slightly shifted to a higher degree for the acid-etched surface compared with the machined surface may indicate that the acid-etched surface is covered by a thicker oxidized layer. This could explain its greater UV-responsive physicochemical changes for the acid-etched surface.

The level of hydrocarbon, and not hydrophilicity level, strongly correlated with rates of protein adsorption and cell attachment. In light of this finding, the amount of hydrocarbon adsorbed on TiO₂ at the time of implantation seems to be crucial in determining the initial affinity level for osteoblasts and consequently manifesting the distinction in bone morphogenesis in vivo and determining the degree of bone-titanium integration. The levels of protein adsorption and the number of cells attached on control titanium surfaces remained low compared with those on UV-treated surfaces even after prolonged incubation suggesting credible long-term effects caused by the initial biological environment. Currently used titanium implants for clinical and experimental use are found to contain hydrocarbons contaminated. Progressive accumulation of organic molecules particularly those with a carbonyl moiety onto titanium surfaces is considered unavoidable under ambient conditions. This may explain the relatively low bone-titanium contact results (45-75%) as described earlier. The present invention demonstrated that bone-titanium contact can be increased up to nearly 100% by treating titanium implants with UV light.

The proteins and osteoblastic cells tested are negatively charged. When oxygen-containing hydrocarbons covering of TiO₂ surfaces are removed by UV light treatment, Ti⁴⁺ sites are exposed. This may promote the interaction between the proteins and cells and such cationic sites. The generation of a bio-affinity TiO₂ surface associated with the photodecomposition of hydrocarbons is schematically proposed in FIG. 6M.

Many efforts have been made in osseous implant therapy both in dental and orthopedic fields to minimize failure rate, shorten morbidity and maximize post-operation functionality. One issue is that the implant placement for rehabilitation faces bone which is impaired in regenerative potential and metabolic activity which specifically delay and hinder the process of bone-titanium integration. Another issue is that the use of acrylic bone cement in some implant procedures inherently limits the biocompatibility and long-term prediction of implants. There is a trend toward cement-free implantation to avoid bone cement complications. These epidemiological, surgical, and societal issues strongly justify efforts to develop a new implant therapy with greater versatility and better lifetime predictability. The major benefit obtained from the physicochemical modification of titanium using UV light presented in the present invention is the 3-time-stronger anchorage of the implants at the early healing stage which corresponds to a 4-time acceleration in the establishment of bone-titanium integration. Given that the UV effect on enhancing osseointegration capacity was demonstrated on both the machined and acid-etched surfaces, the application of this technology is much expected to be extendable to other surface types that comprise a majority of the currently available titanium implants. This technology has immediate and extensive applications in dental, facial and orthopedic implant therapies, because of its simplicity, high efficacy and low-cost.

Provided herein is a method for functionalizing an implant, comprising (1) providing an implant surface, and (2) treating the implant surface thereby causing the surface to be electro-positively charged or enhancing the electro-positive charge on the surface. In some embodiments, the method causes or enhances electro-positive charge under the physiological condition. The physiological condition can have pH value of about 7. In some embodiments, the method causes or enhances electro-positive charge at a pH lower than 7 or at a pH higher than 7.

In one embodiment, the implant has a titanium surface. In one embodiment, the implant further comprises a carrier material which can be metallic or non-metallic. The titanium surface comprises TiO₂. In some embodiments, the treated surface is substantially free of hydrocarbon. In some embodiments, the treated surface comprises a titanium oxide cation.

The atomic percentage of carbon on titanium surfaces can be reduced to lower than 20% as opposed to approximately higher than 50% on the untreated or old titanium surfaces.

The implant surface is treated by applying ultraviolet (UV) light to it. The UV light can be applied by a UV lamp. The UV light can be of a wave-length of about 10 nm to about 400 nm. In some embodiments, the UV light can be of a wave-length of about 170 nm to about 270 nm or about 340 nm to about 380 nm. In some embodiments, the surface is treated by applying a combination of a UV light of a wave-length of about 170 nm to about 270 nm and a UV light of wave-length of about 340 nm to about 380 nm.

The UV light intensity can have a wide range. For example the UV light intensity can be in the range between 0.001 mW/cm² and 100 mW/cm². In some embodiments, the UV light can be of an intensity of about 0.1 mW/cm² or about 2 mW/cm².

The treatment with UV light can be over a period of time up to 48 hours, e.g. 30 second, 1 minute, 5 minutes, 15 minutes, 30 minutes, 1 hour, 5 hours, 10 hours, 24 hours, 36 hours, and 48 hours.

In one embodiment, the method further comprises processing the implant surface prior to treating the implant surface. The implant surface can be processed by a physical process or a chemical process. The physical process can be machining or sandblasting. The chemical process can be etching by acid or base. The acid can be sulfuric acid. The newly processed surface can have electro-positive charge. The UV treatment can enhance the processed surface's electro-positiveness.

In one embodiment, the treated surface can attract proteins and cells at an enhanced rate. As used herein “enhanced rate” means the rate at which the treated implant surface attracts cells or proteins is higher than that of the corresponding untreated implant surfaces. The untreated implant surfaces include newly processed surfaces and “old” surfaces which have been processed and aged for a period of time such as 1 day, 3 days, one week, two weeks, 3 weeks, 4 works, etc. The enhanced rate can be 5%, 10%, 15%, 20%, 25%, 30%, 35%, 40%, 45%, 50%, 60%, 70%, 80%, 90%, 100%, 200%, 300%, 400% etc. higher than the rate at which the corresponding untreated surfaces attract proteins or cells.

As used herein, the term “enhance” can be used interchangeably with the term ‘improve” or “increase.” Enhancing means being made faster, stronger, or higher in an amount.

The protein can be bovine serum albumin, fraction V, and bovine plasma fibronectin. The cell can be human mesenchymal stem cell and osteoblastic cell. The protein or cells can attach to the treated implant surface directly, e.g. without a bridging divalent cation. In one embodiment, the treated titanium surface does not comprise a divalent cation such as Ca²⁺, Mg²⁺, Zn²⁺, etc.

The treated implant surface can enhance tissue-implant integration and/or bone-implant integration at the implant site. The treated implant surface has improved bone-forming capacity over the non-treated implant surface. The treated surface can enhance tissue-implant integration, bone-implant integration, or bone-forming activity over its corresponding untreated surfaces by a percentage such as 5%, 10%, 15%, 20%, 25%, 30%, 35%, 40%, 45%, 50%, 60%, 70%, 80%, 90%, 100%, 200%, 300%, 400%, 500%, etc.

The treated implant surface is capable of any of the following: increasing adsorption of protein, increasing osteoblast migration, increasing attachment of osteoblasts, facilitating osteoblast spread, increasing proliferation of osteoblast, and promoting osteoblastic differentiation, over untreated surfaces. Each of the various activities can be increased by a percentage such as 5%, 10%, 15%, 20%, 25%, 30%, 35%, 40%, 45%, 50%, 60%, 70%, 80%, 90%, 100%, 200%, 300%, 400%, 500%, etc.

Provided herein is an implant which comprises a surface which is functionalized according to the method described above. In one embodiment, the medical implant comprises a titanium surface. The titanium surface comprises TiO₂ bearing positive charge. In one embodiment, the titanium surface is substantially free of hydrocarbon.

The implant further comprises a carrier material. In one embodiment, the carrier material is metallic. In one embodiment, the carrier material is non-metallic.

The implant surface can attract proteins or cells at an enhanced rate. As used herein “enhanced rate” means the rate at which the implant surface attracts cells or proteins is higher than that of surfaces without positive charge or less positive charge. The enhanced rate can be 5%, 10%, 15%, 20%, 25%, 30%, 35%, 40%, 45%, 50%, 60%, 70%, 80%, 90%, 100%, 200%, 300%, 400%, 500%, etc, higher than the rate of the corresponding surfaces without positive charge or less positive charge.

The implant surface can attract a protein such as bovine serum albumin, fraction V, and bovine plasma fibronectin. The implant surface can attract a cell such as human mesenchymal stem cell and osteoblastic cell.

The implant surface is capable of enhancing tissue-implant integration and/or bone-implant integration. The implant surface can enhance tissue-implant integration, bone-implant integration, or bone-forming activity over surfaces without positive charge or less positive charge by a percentage such as 5%, 10%, 15%, 20%, 25%, 30%, 35%, 40%, 45%, 50%, 60%, 70%, 80%, 90%, 100%, 200%, 300%, 400%, 500%, etc.

The implant surface is capable of any of the following: increasing adsorption of protein, increasing osteoblast migration, increasing attachment of osteoblasts, facilitating osteoblast spread, increasing proliferation of osteoblast, and promoting osteoblastic differentiation, over surfaces without positive charge or less positive charge. Each of the various activities can be increased by a percentage such as 5%, 10%, 15%, 20%, 25%, 30%, 35%, 40%, 45%, 50%, 60%, 70%, 80%, 90%, 100%, 200%, 300%, 400%, 500%, etc.

Provided herein are novel titanium surfaces that exhibit an enhanced bioactivity, attracting proteins and biological cells. The titanium surfaces are electro-positively charged and are created by exposing the fresh layer of titanium and/or treating the surface with ultraviolet (UV) light. The exposure of the fresh titanium layer includes newly processing the surface, such as machining, etching, sandblasting, and a combination of these, and also re-processing old surfaces. The present invention has immediate and broad applications in dental and orthopedic implants as well as in the fields of bone regenerative therapy and bone engineering because it is simple, highly effective, and inexpensive.

It is found that UV light treatment of titanium enhances its osteoconductive capacity. The effects of UV treatment of titanium on various in vitro behaviors and functions of osteoblasts on the titanium substrate and in vivo potential of bone-titanium integration and factors on UV-treated titanium surfaces responsible for the enhanced osteoconductivity are examined

Provided herein is a method for enhancing titanium's osteoconductive capacity and titanium surfaces with enhanced osteoconductive capacity made using the method. Machined and acid-etched titanium samples were treated with UV for various time periods up to 48 hours. For both surfaces, UV treatment increased the rates of attachment, spread, proliferation, and differentiation of rat bone marrow-derived osteoblasts as well as the capacity of protein adsorption by up to threefold. In vivo histomorphometry in the rat model revealed that new bone formation occurred extensively on UV-treated implants with virtually no intervention by soft tissue maximizing bone-implant contact up to nearly 100% at week 4 of healing.

An implant biomechanical test revealed that UV treatment accelerated the establishment of implant fixation 4 times. The rates of protein adsorption and cell attachment strongly correlated with the UV dose-responsive atomic percentage of carbon on TiO₂, but not with the hydrophilic status. The data indicated that UV light pretreatment of titanium substantially enhances its osteoconductive capacity in association with UV-catalytic progressive removal of hydrocarbons from the TiO₂ surface suggesting a photo-functionalization of titanium enabling more rapid and complete establishment of bone-titanium integration.

Ultraviolet (UV) light treatment of titanium surfaces markedly increased their osteoconductive capacity. New bone formation occurred extensively on UV-treated implants with virtually no intervention by soft tissue, maximizing bone-implant contact up to nearly 100% at week 4 of healing, whereas the bone-implant contact of untreated implants remained approximately 50%. UV treatment enhanced the strength of bone-titanium integration over 3 times at week 2 of healing. The UV-treated surface offered osteoblast-affinity environment, as demonstrated by enhanced attachment, spread, proliferation, and differentiation of osteoblasts, as well as increased protein adsorption. The rates of protein adsorption and cell attachment strongly correlated with the UV dose-responsive atomic percentage of carbon on TiO₂, but not with the hydrophilic status. This UV-mediated enhancement of titanium bioactivity was demonstrated on different surface topographies of machined and acid-etched surfaces. Therefore it is provided herein a method of photofunctionalization of titanium enabling more rapid and complete establishment of bone-titanium integration.

### Medical Implants

The medical implants can be metallic implants or non-metallic implants. In some embodiments, the medical implants are metallic implants such as titanium implants, e.g., titanium implants for replacing missing teeth (dental implants) or fixing diseased, fractured or transplanted bone. Other exemplary metallic implants include, but are not limited to, titanium alloy implants, chromium-cobalt alloy implants, platinum and platinum alloy implants, nickel and nickel alloy implants, stainless steel implants, zirconium, chromium-cobalt alloy, gold or gold alloy implants, and aluminum or aluminum alloy implants.

The metallic implants described herein include titanium implants and non-titanium implants. Titanium implants include tooth or bone replacements made of titanium or an alloy that includes titanium. Titanium bone replacements include, e.g., knee joint and hip joint prostheses, femoral neck replacement, spine replacement and repair, neck bone replacement and repair, jaw bone repair, fixation and augmentation, transplanted bone fixation, and other limb prostheses. None-titanium metallic implants include tooth or bone implants made of gold, platinum, tantalum, niobium, nickel, iron, chromium, titanium, titanium alloy, titanium oxide, cobalt, zirconium, zirconium oxide, mangnesium, magnesium, aluminum, palladium, an alloy formed thereof, e.g., stainless steel, or combinations thereof Some examples of alloys are titanium-nickel allows such as nitanol, chromium-cobalt alloys, stainless steel, or combinations thereof In some embodiments, the metallic implant can specifically exclude any of the aforementioned metals.

Non-metallic implants include, for example, ceramic implants, calcium phosphate or polymeric implants. Useful polymeric implants can be any biocompatible implants, e.g., bio-degradable polymeric implants. Representative ceramic implants include, e.g., bioglass and silicon dioxide implants. Calcium phosphate implants includes, e.g., hydroxyapatite, tricalcium phosphate (TCP). Exemplary polymeric implants include, e.g., poly-lactic-co-glycolic acid (PLGA), polyacrylate such as polymethacrylates and polyacrylates, and poly-lactic acid (PLA) implants.

In some embodiments, the implant comprises a metallic implant and a bone-cement material. The bone cement material can be any bone cement material known in the art. Some representative bone cement materials include, but are not limited to, polyacrylate or polymethacrylate based materials such as poly(methyl methacrylate) (PMMA)/methyl methacrylate (MMA), polyester based materials such as PLA or PLGA, bioglass, ceramics, calcium phosphate-based materials, calcium-based materials, and combinations thereof In some embodiments, the medical implant can include any polymer described below. In some embodiments, the medical implant described herein can specifically exclude any of the aforementioned materials.

The term “osteoconductive capacity” or “osteoconductivity” refers to bone forming capacity. It also refers to the ability that imparts enhanced bone integration capability to a medical implant. Bone integration capability refers to the ability of a medical implant to be integrated into the bone of a biological body. Tissue integration capacity refers to the ability of a medical implant to be integrated into the tissue of a biological body.

### UV Irradiation

As used herein, the term “applying UV” can be used interchangeably with the term “light activation,” “light radiation,” “light irradiation,” “UV light activation,” “UV light radiation,” or “UV light irradiation.” The radiation having a wavelength from about 400 nm to 10 nm is generally referred to as ultraviolet (UV) light.

The medical implants can be radiated with or without sterilization. To one of ordinary skill in the art, the medical implants can be sterilized during the process of UV radiation.

In one aspect of the present invention, it is provided a facility or device for radiating medical implants. In one embodiment, the facility or device includes a chamber for placing medical implants, a source of high energy radiation and a switch to switch on or turn off the radiation. The facility or device may further include a timer. In some embodiments, the facility or device can further include a mechanism to cause the medical implants or the UV radiation source to turn or spin for full radiation of the implants. Alternatively, the chamber for placing medical implants can have a reflective surface so that the radiation can be directed to the medical implants from different angles, e.g., 360 degree angle. In some embodiments, the facility or device may include a preservation mechanism of the enhanced bone-integration capability, e.g., multiple irradiation of light, radio-lucent implant packaging, packing and shipping.

### Medical Uses

The medical implants provided herein can be used for treating, preventing, ameliorating, correcting, or reducing the symptoms of a medical condition by implanting the medical implants in a mammalian subject. The mammalian subject can be a human being or a veterinary animal such as a dog, a cat, a horse, a cow, a bull, or a monkey.

Representative medical conditions that can be treated or prevented using the implants provided herein include, but are not limited to, missing teeth or bone related medical conditions such as femoral neck fracture, missing teeth, a need for orthodontic anchorage or bone related medical conditions such as femoral neck fracture, neck bone fracture, wrist fracture, spine fracture/disorder or spinal disk displacement, fracture or degenerative changes of joints such as knee joint arthritis, bone and other tissue defect or recession caused by a disorder or body condition such as, e.g., cancer, injury, systemic metabolism, infection or aging, and combinations thereof

In some embodiments, the medical implants provided herein can be used to treat, prevent, ameliorate, or reduce symptoms of a medical condition such as missing teeth, a need for orthodontic anchorage or bone related medical conditions such as femoral neck fracture, neck bone fracture, wrist fracture, spine fracture/disorder or spinal disk displacement, fracture or degenerative changes of joints such as knee joint arthritis, bone and other tissue defect or recession caused by a body condition or disorder such as cancer, injury, systemic metabolism, infection and aging, limb amputation resulting from injuries and diseases, and combinations thereof.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

### EXAMPLES

### Titanium Samples, Surface Analysis and UV Light Treatment

Two surface types of commercially pure titanium were prepared for cylindrical implants (1 mm in diameter, 2 mm in length) and disks (20 mm in diameter, 1.5 mm in thickness). One had a machined surface, turned by a lathe and other was acid-etched with 67% H₂SO₄ at 120° C. for 75 seconds. Additionally, machined surfaces and sandblasted surfaces were prepared. All surfaces were examined by spectrophotometer (UV-2200A, Shimadzu, Tokyo, Japan) and X-ray diffraction (XRD) (XRD-6100, Shimadzu, Tokyo, Japan) to determine their optical property and crystalline structure, respectively. Hydrophilic status of the titanium surfaces was examined by the contact angle of 1 μl H₂O droplet measured by a contact angle meter (CA-X, Kyowa Interface Science, Tokyo, Japan). All procedures were performed in a class 10 clean room under controlled conditions of 20° C. and 46% humidity.

The chemical composition on titanium surfaces were evaluated by electron spectroscopy for chemical analysis (ESCA). ESCA was performed using an X-ray photoelectron spectroscopy (XPS) (ESCA3200, Shimadzu, Tokyo, Japan) under high vacuum conditions (6×10⁻⁷ Pa). Titanium disks and cylindrical implants treated UV light for various periods of time up to 48 hours under ambient conditions were compared with untreated control ones for surface properties and biological potential. UV light treatment was performed using a 15 W bactericidal lamp (Toshiba, Tokyo, Japan); intensity; ca. 0.1 mW/cm² (UVA: λ=360±20 nm) and 2 mW/cm² (UVC: λ=250±20 nm).

A separate use of UVA and UVC was also tested for activation capability for titanium surfaces.

UV-treatment can be performed under a normal ambient air condition, without any atmosphere set-up, such as vacuum or adding inert gas. It is postulated that UV treatment of titanium or titanium-containing metals results in the excitement of electrons from valence band to conduction band of titanium atoms, which results in the creation of positive hole in the superficial layer of titanium and generate the electropositive charge on its surface. To make this electron excitement happen, UV light energy of 3.2 eV is needed, which corresponds to approximately 365 nm wavelength, referred to as UVA. In contrast, direct hydrocarbon decomposition is enforced by UVC at its peak wavelength of lower than 260 nm. This carbon removal facilitates the penetration of UVA to the titanium surface and increases the efficiency of the generation of electropositiveness and eventually expedites and enhance the exposure of the generated electropositive change.

Without being bound by any theories, a combination of UVA (about 340 nm to about 380 nm) and UVC (about 170 nm to about 270 nm) was used.

### Measurement of Protein Adsorption

Bovine serum albumin, fraction V (Pierce Biotechnology, Inc., Rockford, Ill.) and bovine plasma fibronectin (Sigma-Aldrich, St. Louis, Mo) were used as model proteins. Three hundred ml of protein solution (1 mg/ml protein/saline) was spread over a Ti disk using a pipette. After several different periods of incubation (e.g. 2, 6, 24, or 72 hour of incubation) in sterile humidified condition at 37° C., nonadherent protein was removed and washed twice using saline with 0.9% sodium chloride. Aliquots (200 μl) of the initial and removed solutions were mixed with 200 μl of microbicinchoninic acid (Pierce Biotechnology, Inc., Rockford, Ill.) and incubated at 37° C. for 60 minutes. The amount of protein was quantified using a microplate reader at 562 nm.

### Human Mesenchymal Stem Culture

Human mesenchymal stem cells (MSCs) (Poietics, Cambrex Bio Science Walkersville, East Rutherford, N.J.) were cultured in MSC growth medium that consisted of MSC basal medium and growth supplements (SingleQuots). The growth supplements contained fetal bovine serum (FBS), L-glutamine and penicillin/streptomycin. Cells were incubated in a humidified atmosphere with 95% air, 5% CO₂ at 37° C. At 80% confluency of the last passage, cells were detached using 0.25% trypsin-1 mM EDTA-4Na and seeded onto Ti disks at a density of 3×10⁴ cells/cm². The culture medium was renewed every three days.

### Osteoblastic Cell Culture

Bone marrow cells isolated from the femur of 8-week-old male Sprague-Dawley rats were placed into alpha-modified Eagle's medium supplemented with 15% fetal bovine serum, 50 μg/ml ascorbic acid, 10 mM Na-β-glycerophosphate, 10⁻⁸M dexamethasone and antibiotic-antimycotic solution. Cells were incubated in a humidified atmosphere of 95% air, 5% CO₂ at 37° C. At 80% confluency, cells were detached using 0.25% trypsin-1 mM EDTA-4Na and seeded onto machined or acid-etched titanium disks with and without UV treatment at a density of 3×10⁴ cells/cm². The culture medium was renewed every three days.

### Migration Assay

Migration of human MSCs to Ti surfaces was examined using dual-chamber migration assay (345-024K, Trevigen, Gaithersburg, Md.). Cells were seeded into the top chamber in the culture medium. A Ti disk was placed at the bottom of the lower chamber. The percentage of cells that penetrated into the lower chamber after 3 hours of incubation at 37° C. through a polyester membrane with 8-μm diameter pores was analyzed using the plate reader after staining with calcein-AM.

### Cell Attachment, Density and Proliferation Assays

Initial attachment of cells was evaluated by measuring the quantity of the cells attached to titanium substrates after 3 hours and 24 hours of incubation. In addition, the propagated cells were quantified as cell density at culture days of 2 and 5. These quantifications were performed using WST-1 based colorimetry (WST-1, Roche Applied Science, Mannnheim, Germany). The culture well was incubated at 37° C. for 4 hours with 100 μl tetrazolium salt (WST-1) reagent. The amount of formazan product was measured using an ELISA reader at 420 nm. Further, the cells were stained with calcein AM for the observation under a fluorescent microscope to confirm the cell density results.

The proliferative activity of the cells was measured by BrdU incorporation during DNA synthesis. At day 2 of culture, 100 μl of 100 mM BrdU solution (Roche Applied Science, Mannheim, Germany) was added to the culture wells and incubated for 10 hours. After trypsinizing the cells and denaturing the DNAs, the cultures were incubated with anti-BrdU conjugated with peroxidase for 90 minutes and reacted with tetramethylbenzidine for color development. Absorbance at 370 nm was measured using an ELISA reader (Synergy HT, BioTek Instruments, Winooski, Vt.).

### Cell Morphology and Morphometry

Confocal laser scanning microscopy was performed to examine the morphology and cytoskeletal arrangement of human MSCs. After 3 hour of culture, the cells were fixed in 10% formalin, and stained using a fluorescent dye, rhodamine phalloidin (actin filament red color, Molecular Probes, Oreg.). The cultures were also immunochemically stained with mouse anti-paxillin monoclonal antibody (Abcam, Cambridge, Mass.), followed by the adding of FITC-conjugated anti-mouse secondary antibody (Abcam, Cambridge, Mass.). The cell area, perimeter, and Feret's diameter were quantitatively assessed using an image analyzer (ImageJ, NIH, Bethesda, Md.).

After 3 hour of culture osteoblasts were fixed in 10% formalin, and stained using fluorescent dyes, DAPI (nuclei blue color, Vector, Calif.) and rhodamine phalloidin (actin filament red color, Molecular Probes, Oreg.). Confocal laser scanning microscopy was used to examine cell morphology and cytoskeletal arrangement. Quantitative assessment for cell area, perimeter and Feret's diameter was performed using an image analyzer (Image J, NIH, Bethesda, Md.).

### Alkaline Phosphatase (ALP) Activity

The ALP activity of cultured osteoblasts was examined by culture area- and colorimetry-based assays. Cultured osteoblastic cells were washed twice with Hanks' solution, and incubated with 120 mM Tris buffer (pH 8.4) containing 0.9 mM naphthol AS-MX phosphate and 1.8 mM fast red TR for 30 min at 37° C. The ALP-positive area on the stained images was calculated as [(stained area/total dish area)×100)] (%) using an image analyzing software (Image Pro-plus, Media Cybernetics, Silver Spring, Md., USA). For colorimetry, the culture was rinsed with ddH₂0 and added with 250 μl p-Nitrophenylphosphate (LabAssay ATP, Wako Pure Chemicals, Richmond, Va.), and then incubated at 3TC for 15 minutes. The ALP activity was evaluated as the amount of nitrophenol released through the enzymatic reaction and measured at 405 nm wavelength using ELISA reader (Synergy HT, BioTek Instruments, Winooski, Vt.).

### Mineralization Assay

The mineralization capability of cultured osteoblasts was examined by mineralized nodule area-and calcium colorimetry-based assays. von Kossa stain was utilized to visualize the mineralized nodules of the osteoblastic cells. Cultures were fixed using 50% ethanol/18% formaldehyde solution for 30 min. Cultures were incubated with 5% silver nitrate under UV light for 30 min. Cultures were washed twice with dd H₂O and incubated with 5% sodium thiosulfate solution for 2-5 min. The mineralized nodule area defined as [(stained area/total dish area)×100)] (%) was measured using a image analyzing software (Image Pro-plus, Media Cybernetics, Silver Spring, Md., USA). For colorimetric detection for calcium deposition, cultures were washed with PBS and incubated overnight in 1 ml of 0.5 M HCl solution with gentle shaking. The solution was mixed with o-cresolphthalein complexone in alkaline medium (Calcium Binding and Buffer Reagent, Sigma, St Louis, Mo.) to produce a red calcium-cresolphthalein complexone complex. Color intensity was measured by an ELISA reader (Synergy HT, BioTek Instruments, Winooski, Vt.) at 575 nm absorbance.

### Gene Expression Analysis

Gene expression was semiquantitatively analyzed using reverse transcription-polymerase chain reaction (RT-PCR). Total RNA in the cultures was extracted using TRlzol (Invitrogen, Carlsbad, Calif.) and purification column (RNeasy, Qiagen, Valencia, Calif.). Following DNAse I treatment, reverse transcription of 0.5 μg of total RNA was performed using MMLV reverse transcriptase (Clontech, Carlsbad, Calif.) in the presence of oligo(dT) primer (Clontech, Carlsbad, Calif.). PCR reaction was performed using Taq DNA polymerase (EX Taq, Takara Bio, Madison, Wis.) to detect collagen I, osteopontin, and osteocalcin mRNA using the primer designs and PCR condition established previously. PCR products were visualized on 1.5% agarose gel with ethidium bromide staining. Band intensity was detected and quantified under UV light and normalized with reference to GAPDH mRNA.

### Surgery

Eight-week-old male Sprague-Dawley rats were anesthetized with 1-2% isoflurane inhalation. After their legs were shaved and scrubbed with 10% providone-iodine solution, the distal aspects of the femurs were carefully exposed via skin incision and muscle dissection. The flat surfaces of the distal femurs were selected for implant placement. The implant site was prepared 9 mm from the distal edge of the femur by drilling with a 0.8 mm round burr and enlarged using reamers (#ISO 090 and 100). Profuse irrigation with sterile isotonic saline solution was used for cooling and cleaning. One cylindrical implant was placed into each side of the femurs. Surgical sites were then closed in layers. Muscle and skin were sutured separately with resorbable suture thread. The University of California at Los Angeles (UCLA) Chancellor's Animal Research Committee approved this protocol and all experimentation was performed in accordance with the United States Department of Agriculture (USDA) guidelines of animal research.

### Implant Biomechanical Push-In Test

The implant biomechanical push-in test was used to assess the biomechanical strength of bone-implant integration, and is described elsewhere. Femurs containing a cylindrical implant were harvested and embedded into auto-polymerizing resin with the top surface of the implant level. MicroCT was used to confirm the implants were free from cortical bone support from the lateral and bottom sides of the implant. The testing machine (Instron 5544 electro-mechanical testing system, Instron, Canton, Mass.) equipped with a 2000 N load cell and a pushing rod (diameter=0.8 mm) was used to load the implant vertically downward at a crosshead speed of 1 mm/min. The push-in value was determined by measuring the peak of the load-displacement curve.

### Histological Preparation

The femur containing an acid-etched implant was harvested and fixed in 10% buffered formalin for 2 weeks at 4° C. Specimens were dehydrated in an ascending series of alcohol rinses and embedded in light-curing epoxy resin (Technovit 7200VLC, Hereaus Kulzer, Wehrheim, Germany) without decalcification. Embedded specimens were sawed perpendicular to the longitudinal axis of the cylindrical implants at a site 0.5 mm from the apical end of the implant. Specimens were ground to a thickness of 30 μm with a grinding system (Exakt Apparatebau, Norderstedt, Germany). Sections were stained with Goldner's trichrome stain, and observed via light microscopy.

### Histomorphometry

A 40× magnification lens and a 4× zoom on a computer display were used for computer-based histomorphometric measurements (Image Pro-plus, Media Cybernetics, Silver Spring, Md.). To identify the tissue structure detail, microscopic magnification up to 400× was used. We previously established implant histomorphometry that discriminates between implant-associated bone and non-implant-associated bone. Based on this method, the tissues surrounding implants were divided into two zones as follows: (i) proximal zone, the circumferential zone within 50 μm of the implant surface; and (ii) distant zone, the circumferential zone from 50 μm to 200 μm of the implant surface. The following variables were analyzed:

Bone-implant contact (%)=(sum of the length of bone-implant contact)/(circumference of the implant)×100, where the implant-bone contact was defined as the interface where bone tissue was located within 20 μm of the implant surface without any intervention of soft tissue.

Bone volume in the proximal zone (%)=(bone area in proximal zone)/(area of proximal zone)×100.

Bone volume in the distant zone (%)=(bone area in distal zone)/(area of distant zone)×100.

Soft tissue intervention (%)=(sum of the length of soft tissue intervening between bone and implant)/(sum of the length of bone surrounding an implant)×100.

### Statistical Analyses

Three samples were used for the cell culture studies, except for the evaluation of cell morphometry, which required 10 cell samples. Two-way ANOVA was performed to examine the effects of culture time and Ti surfaces having different ages, with or without UV treatment. If necessary, a post-hoc Bonferroni test was conducted to examine differences among the newly processed, 4-week-old and UV-treated 4-week-old surfaces; p<0.05 was considered statistically significant. If data were available at only one time point, one-way ANOVA was used to determine the differences among the experimental groups. T-test was also used to determine the differences between the untreated control and UV-treated groups. Correlations between the albumin adsorption and cell attachment, and atomic percentage of carbon and H₂O contact angle were examined, and regression formulas were determined by least-squares mean approximation.

### Results

1. Accelerated and Enhanced Protein Adsorption to Newly Processed and UV Light Treated Titanium Surfaces

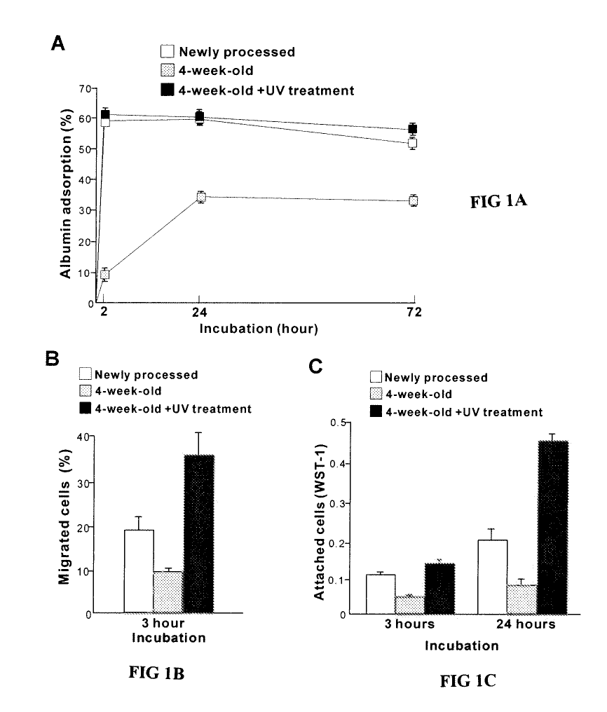

Two-way ANOVA showed that albumin adsorption varied significantly among the experimental groups tested (p<0.01; FIG. 1A); newly processed acid-etched surfaces (immediately after processing), 4-week-old surface (i.e., stored for 4 weeks), UV-treated 4-week-old surface. After 2 hour of incubation, only approximately 10% of albumin incubated in the culture was adsorbed to the 4-week-old Ti surface, while approximately 60% of albumin adsorbed to the fresh surface (p<0.01; Bonferroni). The amount of albumin adsorption was 40% less for the 4-week-old surface than for the new surface even after 72 hour of incubation (p<0.01). The UV light-treated 4-week-old surface showed an albumin adsorption level equivalent to that of the newly processed surfaces after 2 and 24 hours of incubation, and exhibited an even greater level after 72 hours (p<0.05).

2. Stem Cell Migration and Attachment Enhanced on Newly Processed and UV-Treated Titanium Surfaces

The number of human mesenchymal stem cells (MSCs) that had migrated through 8 μm holes varied significantly among culture conditions (p<0.01, 1-way ANOVA; FIG. 1B). The number of cells that migrated to the 4-week-old surface during 3 hour of incubation was 50% of the number observed for the newly processed surface and 25% of the number for the UV-treated 4-week-old surface (p<0.01). The UV-treated 4-week-old surfaces showed a twofold greater cellular migration than the fresh surface (p<0.01).

The number of human MSCs attached to the Ti surfaces increased in the following order: UV-treated 4-week-old surface>newly processed surface>4-week-old surface (p<0.01; 2-way ANOVA; FIG. 1C). The number of cells attached to the 4-week-old surface was less than 50% to the newly processed surface. The UV-treated 4-week-old surface showed a substantially higher (by over 120%) cell attachment than the newly processed surface at 24 hour (p<0.01).

3. Expedited Cell Spread and Cytoskeletal Development on Newly Processed and UV-Treated Ti Surfaces

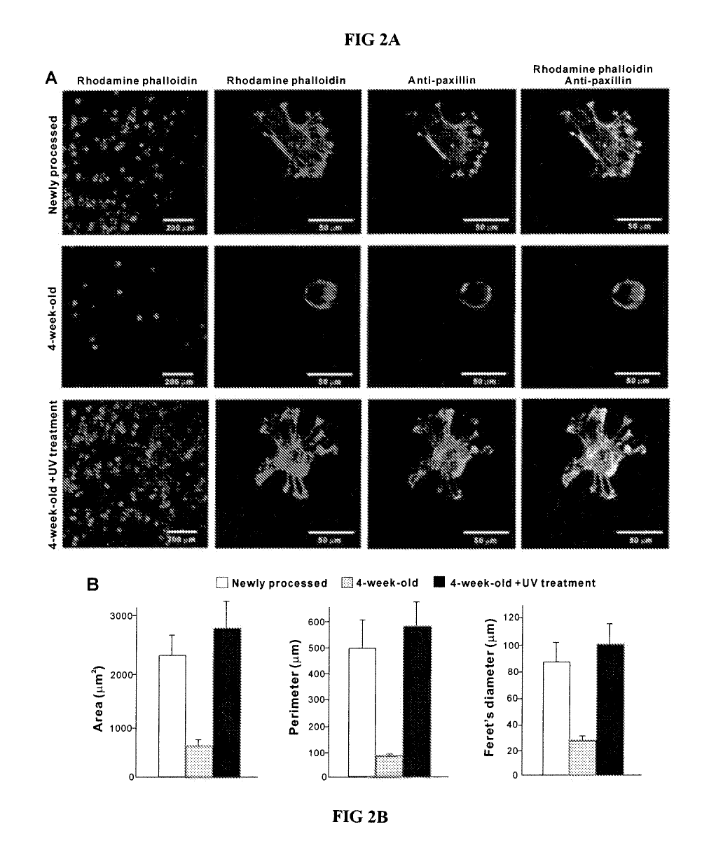

Low magnification images captured after 3 hours of incubation of human MSCs with actin filament (rhodamine phalloidin) stain showed that the number of cells was greatest on the UV-treated 4-week-old surface and lowest on the 4-week-old surface, confirming the result from the cell attachment assays (FIG. 2A). High magnification images with actin stain revealed that cells were clearly larger with their processes spread in multiple directions on the newly processed and UV-treated 4-week-old surfaces, whereas cells remained in rounded form with little cytoskeletal development on the 4-week-old surface. Intensive localization of paxillin, a protein that regulates cell attachment and adhesion, along the cellular configuration was observed in the cells on the newly processed and UV-treated 4-week-old surfaces. In particular, the dense cytoplasmic positive stain was seen in the cells on the UV-treated 4-week-old surface.

Cytomorphometric evaluations of the area, perimeter, and Feret's diameter demonstrated significant differences in these parameters among the three Ti substrates (ANOVA, p<0.01; FIG. 2B). These parameters were 5- to 8-fold greater for the newly processed and the UV-treated surfaces than for the 4-week-old surface (Bonferroni, p<0.01). There were no significant differences between the newly processed and UV-treated surfaces.

4. Enhanced in vivo Bone-Titanium Integration for Newly Processed Titanium and UV-Treated Titanium Surfaces

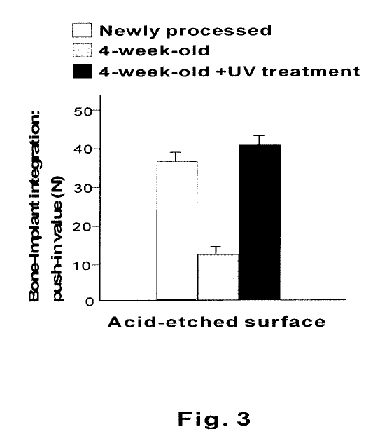

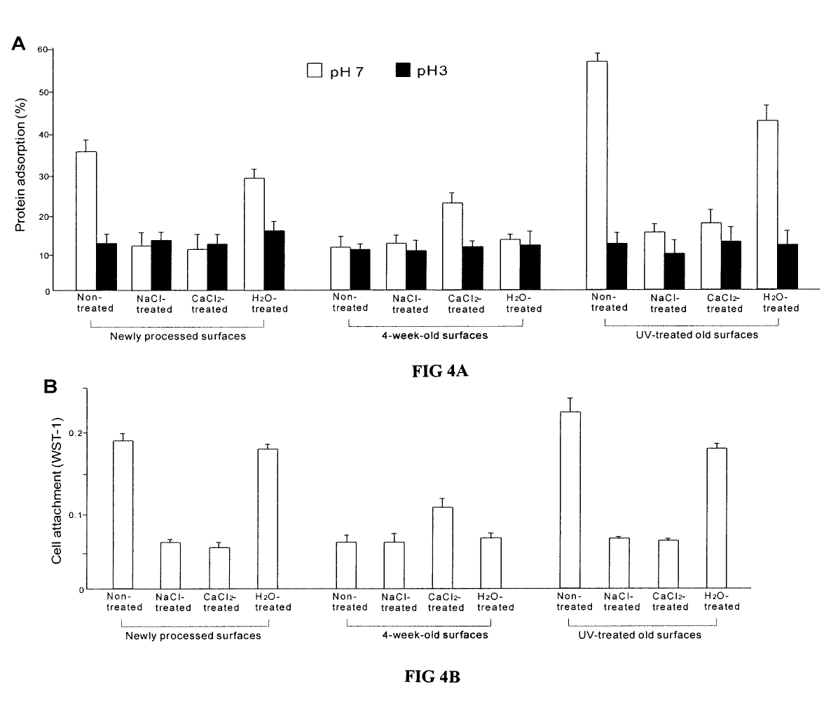

In vivo establishment of implant fixation is the most important factor in determining the clinical capacity of titanium implants as load-bearing devices. In vivo stability of titanium implants was examined using the established biomechanical implant push-in test in a rat model. Cylindrical implants were placed in the rat femur. The strength of bone-titanium integration, measured by push-in value in a rat in vivo model, at the early healing stage of week 2 soared 2.8 times and 3.1 times, respectively, for the newly processed and UV-treated surfaces compared with the 4-week-old surface (p<0.01; FIG. 3).

FIG. 3 shows that enhanced bone-titanium integration for newly processed and UV-treated acid-etched titanium surfaces compared to the 4-week-old surface, evaluated by biomechanical push-in test.

5. Electro-Positively Charged Surfaces of Newly Processed Titanium and UV-Treated Titanium Attract Protein

FIG. 4A shows the albumin adsorption to variously prepared titanium surfaces under different conditions of pH in the medium. Limited amount of albumin adsorbed to the non-treated 4-week-old titanium surfaces at pH 7, with its number between 10-15%. This was a predictable result from the fact that the surfaces of titanium that is ordinarily available, as well as albumin, are negatively charged at this physiologic pH, which prevents the titanium-albumin interaction. Only when the 4-week-old surface was treated with divalent cations, such as CaCl₂, prior to albumin incubation, the albumin adsorption increased. This was explained by that the divalent calcium cations play a bridging role between the negative albumin molecules and titanium surface when deposited to monovalent negative titanium surfaces.

In contrast, as described earlier, the newly processed surface and the UV-treated surface exhibited high adsorption rates of >35% or >55% at pH 7 compared to 4-week-old surface (p<0.01; Non-treated groups in FIG. 4A). However, in the medium prepared with a pH 3, the protein adsorption to those surfaces remained as low as the 4-week-old surfaces. It is known that because the isoelectric pH of albumin is 4.7-4.9, albumin undergoes a neutral-basic transition and becomes positively charged at lower pH values like pH 3, while albumin undergoes a neutral-acidic transition and becomes negatively charged at high pH values like pH 7. These indicate that the newly processed and UV-treated titanium surfaces are positively charged and exhibit differential protein attraction characteristics depending on the environmental pH value. Further, the electro-positive property of these surfaces were confirmed by the tests showing that treating these surfaces with monovalent anions, such as NaCl and CaCl₂ solution, neutralized the existing electro-positiveness of these surfaces and resulted in no increase of the albumin adsorption compared to the baseline level of the non-treated 4-week-old surface.

The newly processed and UV-treated titanium surfaces can maintain electro-positive charge and a low level of surface carbon even at pH 3 and after these ion treatments. This indicates that the surface electropositive charge predominantly regulates the bioactivity of titanium surfaces such as protein adsorption, superseding the effect of superhydrophilicity and carbon level.

6. Electro-Positively Charged Surfaces of Newly Processed Titanium and UV-Treated Titanium Attract Cells

FIG. 4B shows the quantity of human mesenchymal stem cells (MSCs) attached to various titanium surfaces prepared in the same manner as FIG. 4A.

This experiment was performed under the physiologic pH of 7. It was expected that the number of cells attached to the 4-week-old surface was limited because of the repelling force between the titanium surfaces and cells, both are negatively charged. In contrast, a higher number of cells attached to the newly processed and UV-treated old surfaces than to the 4-week-old surface. The numbers of cells attached to the newly processed and UV-treated old surfaces were decreased to the base line level of the non-treated 4-week-old surface after these surfaces were treated with anions such as Cl⁻. Considering the known fact that biological cells are negatively charged, it was demonstrated that surfaces of the newly processed and UV-treated titanium are positively charged and therefore resulted in an enhanced cell-titanium interaction.

Newly processed and UV-treated titanium surfaces can maintain the electro-negative charge and a low level of surface carbon even at pH 3 condition and after these ion treatments. This indicates that surface electropositive charge predominantly regulates the bioactivity of titanium surfaces such as protein adsorption and cell attachment, superseding the effect of superhydrophilicity and carbon level.

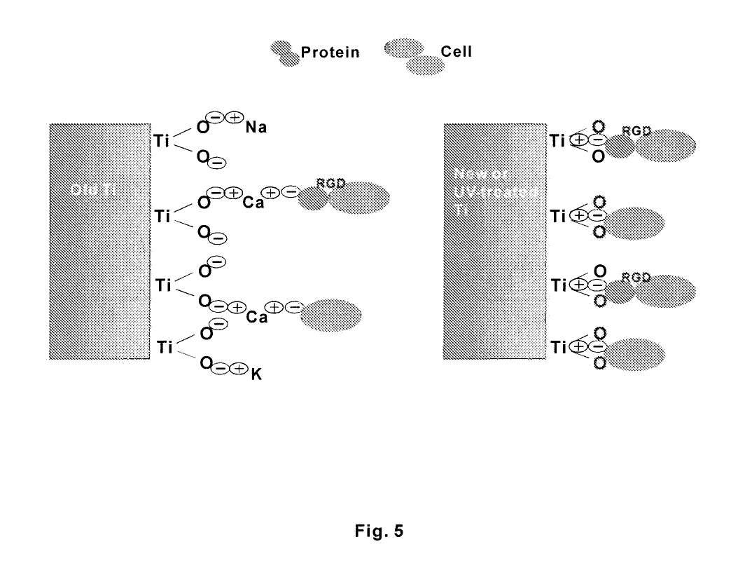

The mechanisms of protein and cellular attachment to titanium surfaces are described in a diagram (FIG. 5). The left side (Old Ti) of the panel shows the mechanism that has been occurring around titanium surface. In the mechanism, the attachment of the cells must be bridged by divalent cations, such as Ca²⁺, in order to adsorb negative proteins and subsequently the cells via RGD sequence of the protein. It is also noted that competitive binding of monovalent cations, such as Na⁺ and K⁺, blocks the anion sites of titanium surface for Ca²⁺ binding. As a result, total number of cells that can be attached to the titanium surface is limited.

The mechanism of the right side (new or UV-treated Ti) presents a novel mechanism based on the present test results in which the titanium surface is converted from cell repellent to cell attractive. Because of the electrostatic positive charge on the newly processed and UV-treated surfaces, negatively charged proteins and cells directly attach to the titanium surface without an aid of divalent cations, resulting in a higher number of cells attached to the surface.

7. Generalization of High Protein and Cell Affinity of Newly Processed and UV-Treated Titanium Surfaces

In addition to the acid-etched titanium surface, machined titanium surfaces and sandblasted titanium surfaces were tested for possible advantages of newly processed surfaces and UV-treated surfaces (FIG. 6A). Four-week-old surfaces showed albumin adsorption of only 20-45% compared with newly prepared surfaces with respective surface groups. UV treatment of the 4-week-old Ti surfaces increased the adsorption rate to a level equivalent to that of newly processed surface by machining or a level higher than the newly processed surfaces by sandblasting (p<0.05).

A similar trend was found in the rate of fibronectin adsorption (FIG. 6B). The rate of adsorption was higher in the order of UV-treated 4-week-old Ti, newly processed Ti and 4-week-old Ti for all three surface topographies tested (p<0.01).

In vivo accomplishment of bone-titanium integration was tested using machined titanium. The UV-treated machined surface exhibited a significant increase of the strength of bone-titanium integration at weeks 2 and 4 of healing (p<0.05; FIG. 6C).

These results indicated that biological advantages of newly processed and UV-treated titanium surfaces are universal for different types of surface processing and effective for different proteins.

8. Photogenerated Superhydrophilicity of Titanium

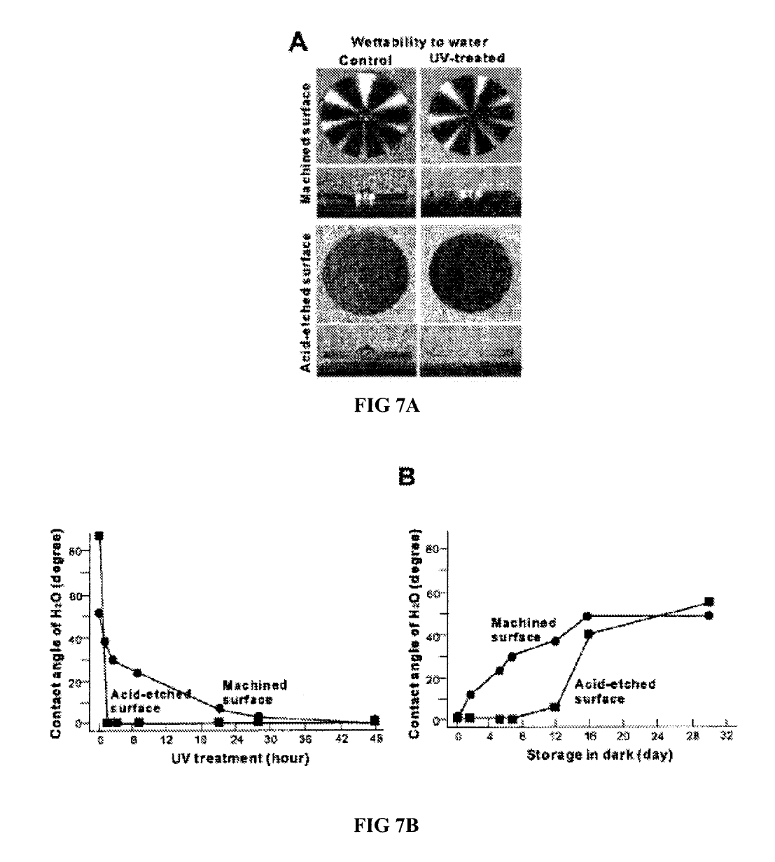

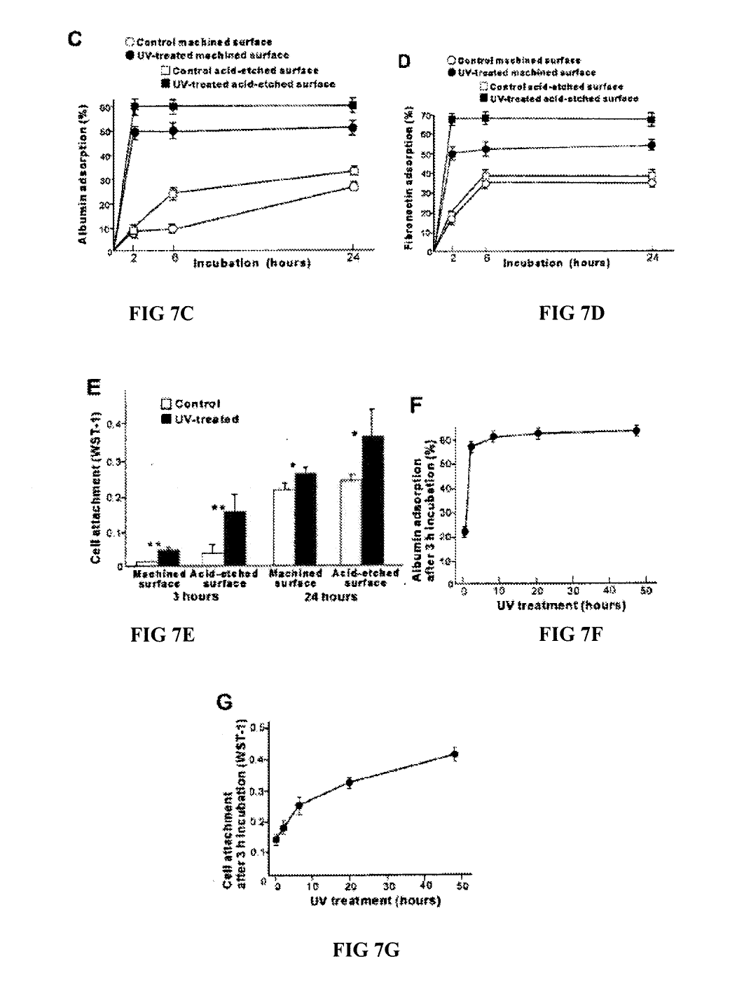

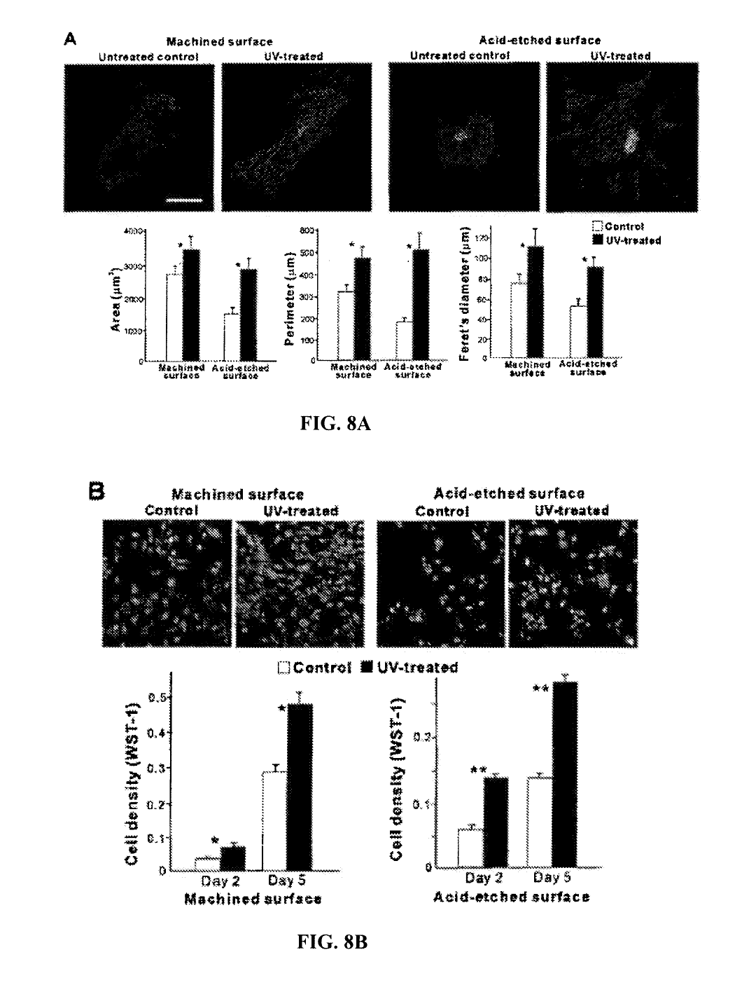

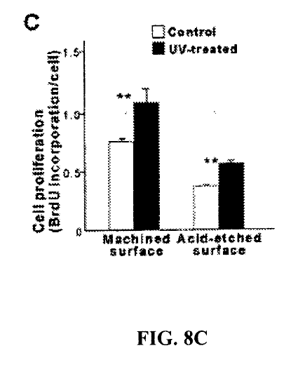

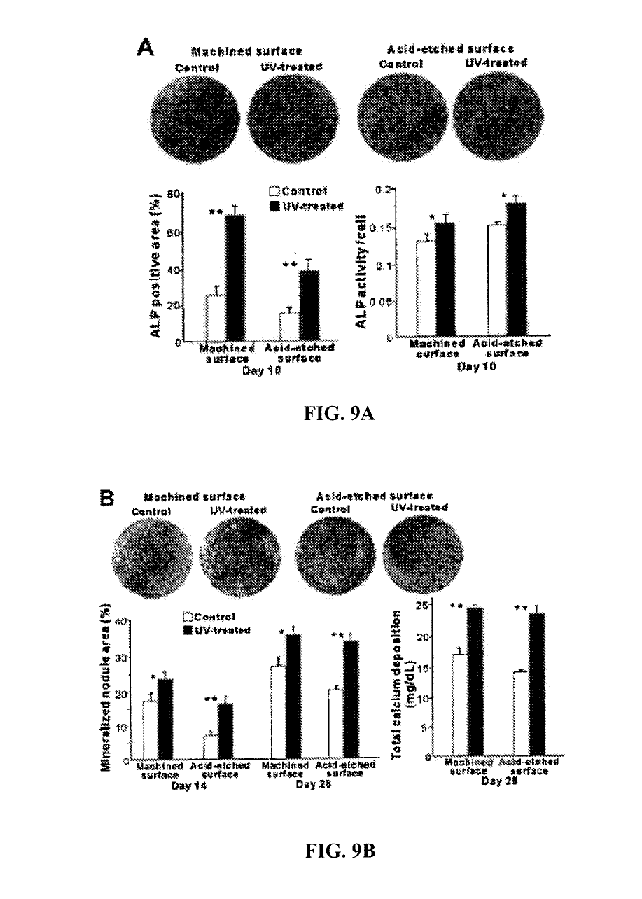

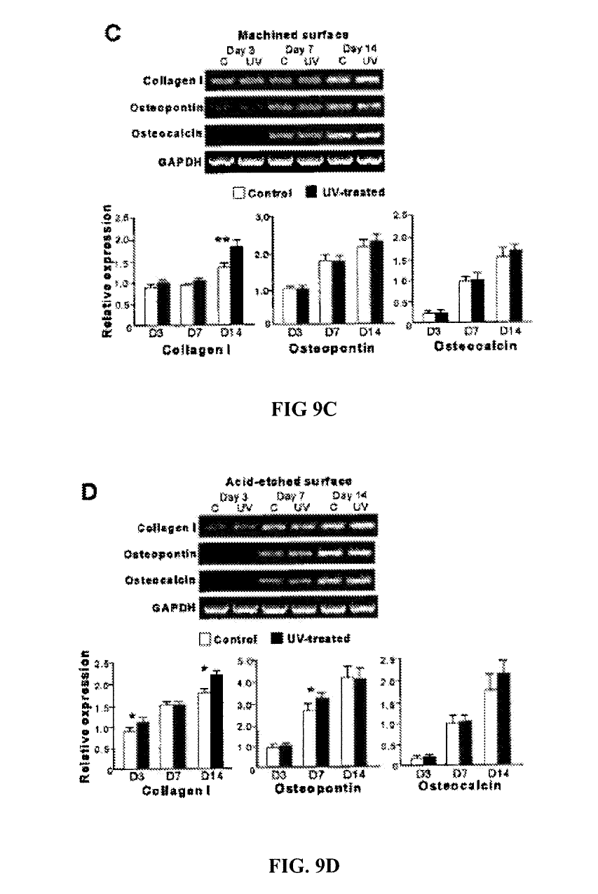

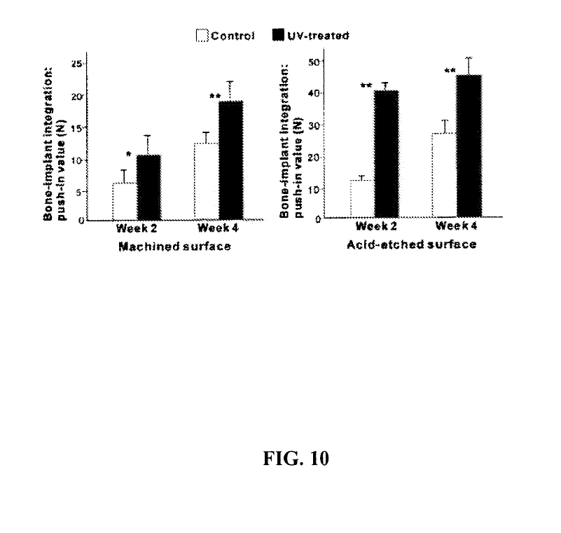

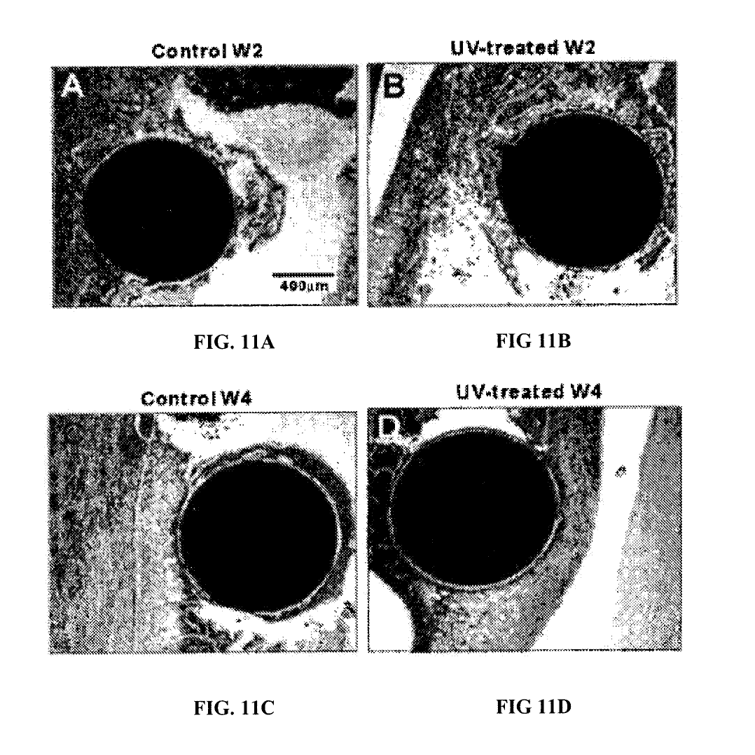

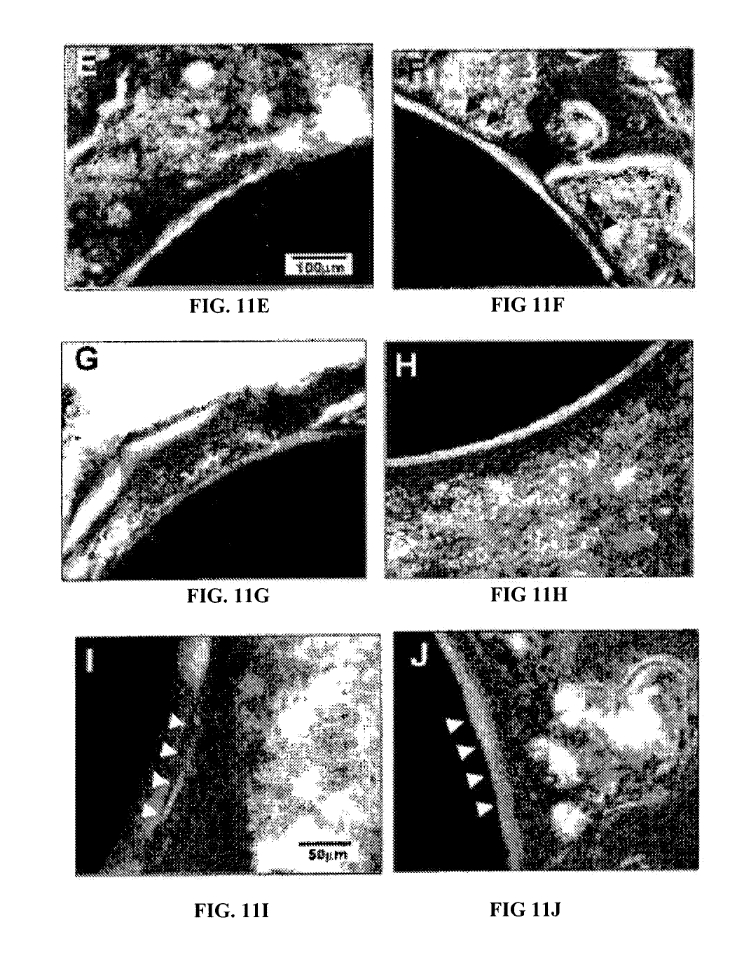

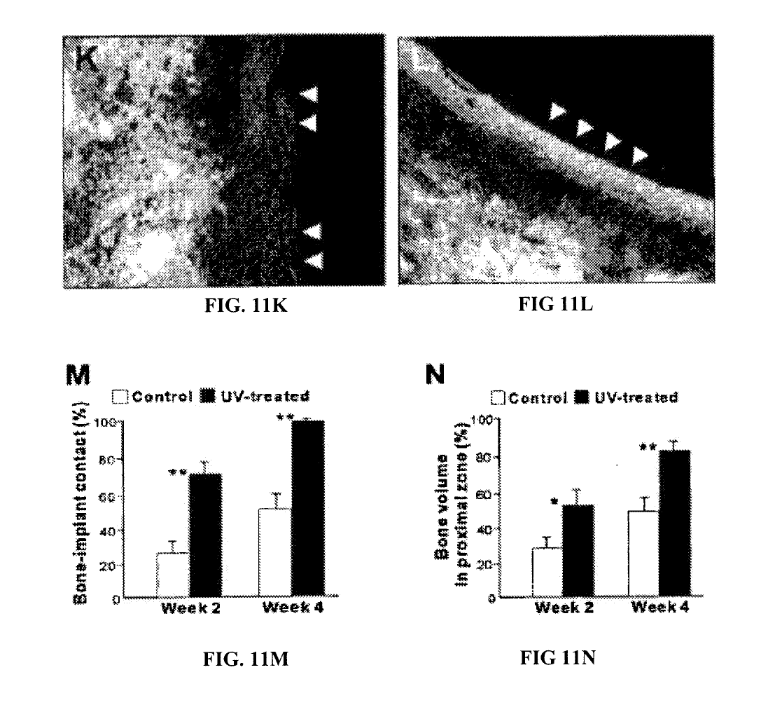

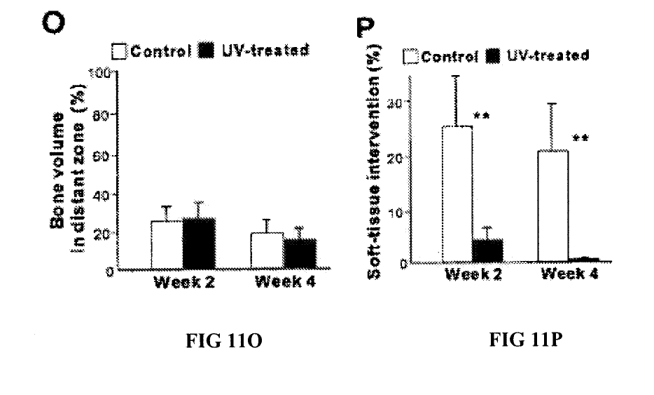

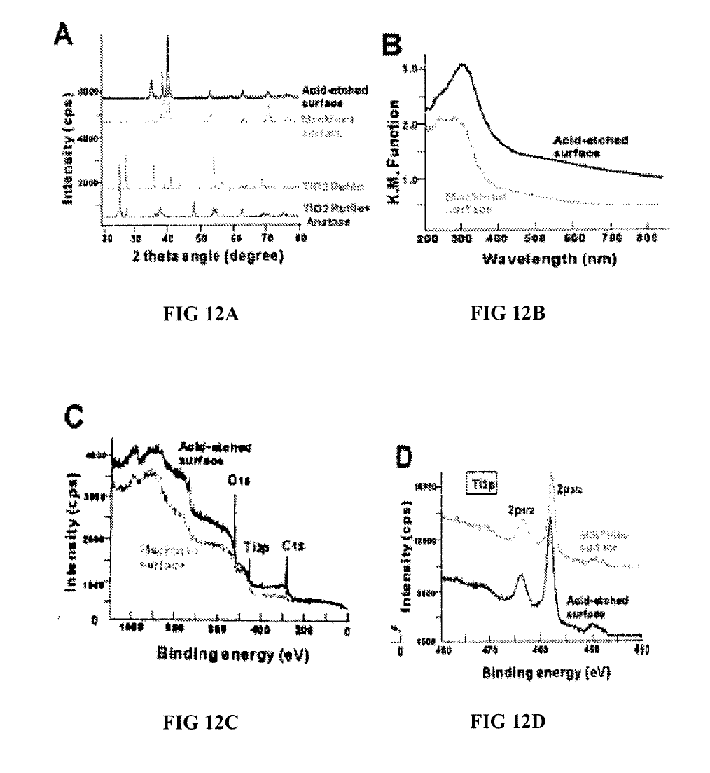

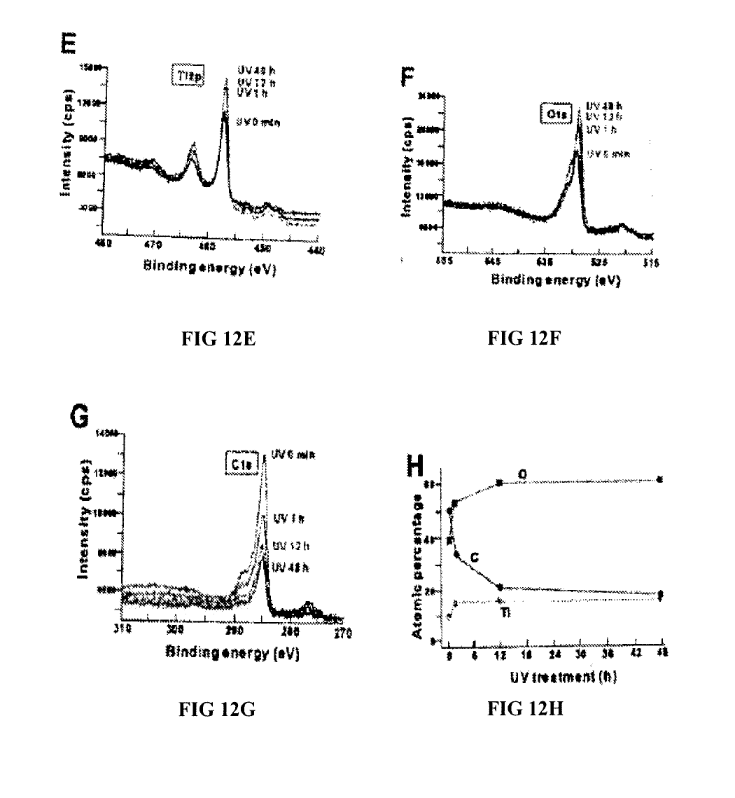

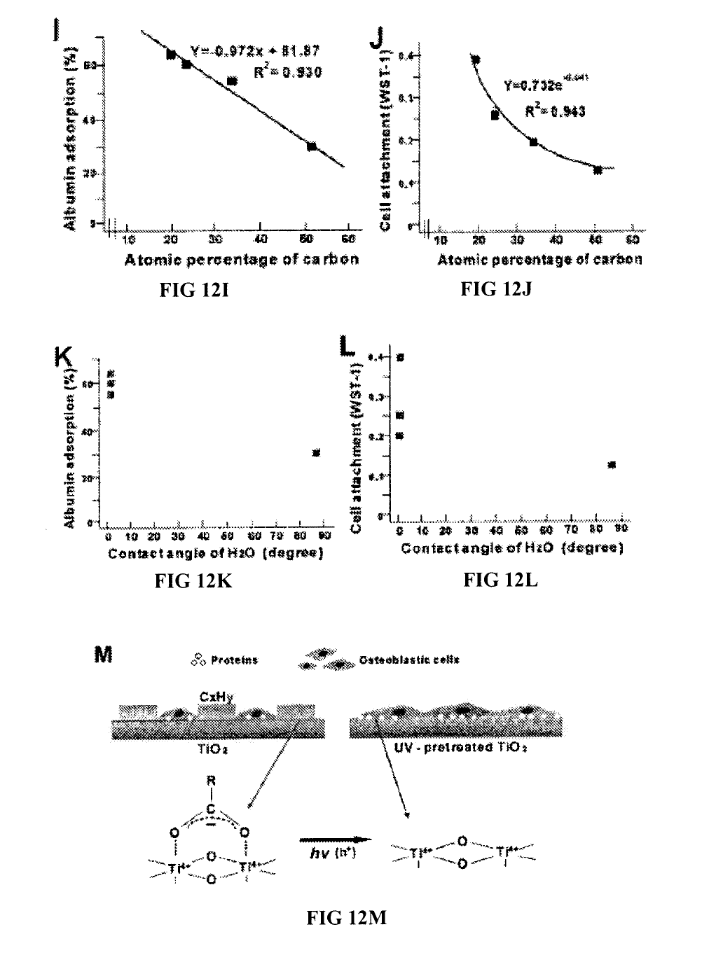

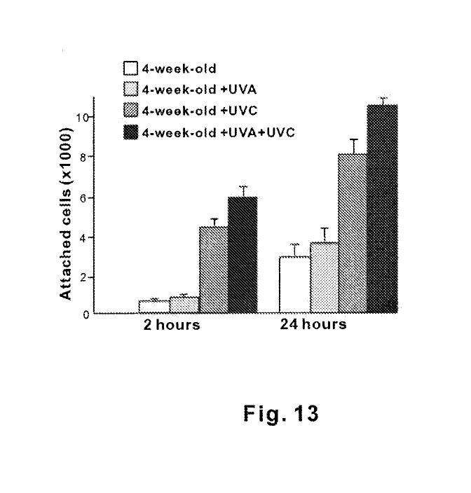

After UV-light treatment of titanium disks for 48 hours, the contact angle of a H₂O droplet, which was 53.5° and 88.4° for the machined and acid-etched surface, respectively, plummeted to 0°, indicating the conversion of hydrophobic surfaces to superhydrophilic surfaces (FIG. 7A). Superhydrophilicity was generated more rapidly on the acid-etched surface. The acid-etched surface required a 1-hour UV treatment, while the machined surface required 48 hours (FIG. 7A). Following 48-hour UV illumination superhydrophilic status was sustained for longer time for the acid-etched surface, with the 0° contact angle of H₂O maintained for 7 days in the dark (FIG. 7B). On the other hand, the superhydrophilicity immediately started to disappear for the machined surface.

9. UV-Enhanced Protein Adsorption Capacity of Titanium

For both surface types (machined and acid-etched), UV treatment accelerated the adsorption of albumin and fibronectin (FIG. 7C, D). For instance, albumin adsorption rate, which was <10% after a 2-hour incubation, increased to 50-60% on titanium surfaces after UV-treated for 48 hours (p<0.01) (FIG. 7C). UV-enhancing effect was greater on the acid-etched surface than on the machined surface for both proteins (p<0.01). The amount of these proteins adsorbed on the untreated surfaces was less than those found on UV-treated surfaces, even after incubation for 24 hours, indicating that UV treatment accelerates and augments protein adsorption by approximately 100% (FIG. 7C, D).

10. Enhanced Attachment of Osteoblasts to UV-Treated Titanium

After 3-hour incubation, the number of the cells attached to UV-treated surfaces was three-to-fivefold greater than to untreated control surfaces for both machined and acid-etched surfaces (FIG. 7E). The UV-induced advantage in cell attachment was present even after 24 hours.

11. UV Dose-Dependency of Biological Effects

To confirm UV-promoted protein adsorption and osteoblast attachment, the UV dose-dependency of the protein adsorption and osteoblast attachment was examined The acid-etched titanium surface was UV-treated for different time periods up to 48 h. UV dosage affected protein adsorption and cell attachment capacities differently (FIG. 7F, G). Increase in the rate of albumin adsorption was rapid, followed by saturation after 1 h of UV treatment. The rate of cell attachment continued to increase significantly with an increase of UV treatment time up to 48 hours (p<0.01).

12. Facilitated Spread and Enhanced Proliferation of Osteoblasts on UV-Treated Titanium

Spread and cytoskeletal development of osteoblasts on the control machined titanium surface appeared to be isotropic along the turned trace from the machining process 3 hours after seeding. Cell processes were rarely developed in these cells. In contrast, the cells on the UV-treated machined surface exhibited philopodia-like cell processes developed in multiple directions (images in FIG. 8A). Cells were clearly larger and the cellular processes stretched to a greater extent on UV-treated acid-etched surfaces than on untreated acid-etched surfaces. Morphometric evaluations for the area, perimeter, and Feret's diameter of the cells showed greater values of these parameters for UV-treated titanium surfaces (histograms in FIG. 8A).

Cell density was consistently greater on UV-treated titanium surfaces than that on untreated surfaces for machined and acid-etched surface types on culture day 2 and day 5 (histograms in FIG. 8B), which was consistent with fluorescent images of the cells after calcein stain (top images in FIG. 8B). BrdU incorporation per cell at day 2 of culture was higher for the UV-treated surfaces, confirming increased osteoblast proliferation (FIG. 8C).

13. Enhanced Osteoblastic Phenotypes on UV-Treated Titanium

At day 10, more than twofold areas in the culture were ALP-positive on UV treated machined and acid-etched surfaces compared with respective control surfaces (top images and lower left histogram in FIG. 9A). In addition, the ALP activity, which was optically quantified and standardized by the number of the cells, was significantly higher on UV-treated titanium surfaces (lower right histogram in FIG. 9A).

At days 14 and 28 of culture, the area of mineralized nodule detected by von Kossa stain was also greater on UV-treated titanium surfaces; This effect was more significant on the acid-etched surface, exhibiting an increase of 120% at day 14 (top images and lower left histogram in FIG. 9B). The total calcium deposition result was consistent with the von Kossa result (lower right histogram in FIG. 9B). RT-PCR analysis showed that, throughout the culture period, the expression of collagen I, osteopontin, and osteocalcin was similar between the cultures with and without UV treatment, or upregulated by <30% on the UV-treated surfaces at some time points (FIG. 9C, D).

14. UV-Enhanced in vivo Implant Fixation

The strength of bone-titanium integration, measured by push-in value, at the early healing stage of week 2 soared 1.8 times and 3.1 times, respectively, for machined and acid-etched surfaces with UV treatment (FIG. 10). At the late stage of healing (week 4), the strength of osseointegration for the UV-treated implants maintained their superiority over the untreated implants by 50% and 60% for the machined and the acid-etched surfaces, respectively.

15. Bone Morphogenesis Around UV-Treated Implant

At week 2, bone tissue with a woven, immature appearance formed in an area relatively distant from the implant surfaces in both the control and the UV-treated acid-etched implants (FIGS. 11A and B). On examining the area adjacent to the implant surface, osteomorphogenic differences were found between the two implants. Bone formation occurred more extensively around UV-treated implant (FIG. 11E, F). Another notable difference was the extent of intervention by soft tissue. Some bone tissues around untreated control implants were associated with soft tissue interposed between the bone and implant (FIG. 11I), which was rarely observed around UV-treated implant (FIG. 11J). At week 4 some parts of the untreated control surface still exhibited fibrous connective tissue intervening between bone and implant (FIG. 11C, G, K), whereas the implants with UV treatment were almost entirely surrounded with directly deposited bone (FIG. 11D, H, L).

Bone histomorphometry revealed that the percentage of bone-implant contact for UV-treated acid-etched implants was consistently greater than for control implants (2.5 times at week 2, 1.9 times at week 4) (FIG. 11M). Bone-implant contact percentage was 98.2% for UV-treated surface. Bone volume in the proximal zone to the implant surface was also consistently greater for UV-treated implants than for control implants (FIG. 11N), whereas there was no UV-induced difference in bone volume in the distant zone, indicating UV-enhanced bone generation specific to the area adjacent to implant surfaces (FIG. 11O). A significant decrease in the percentage of soft tissue intervention by the UV treatment was noted (FIG. 11P). UV-treated surfaces almost completely blocked the soft-tissue from the bone-implant interface at week 4, whereas >20% of bone around untreated surfaces involved soft tissue intervening at titanium interface at weeks 2 and 4.

16. Inverse Correlation Between Carbon Element on Titanium and its Osteoblast and Protein Attractiveness

XRD analyses showed that both machined and acid-etched surfaces did not show any peaks at 25° and 28°, which were typically seen in anatase and rutile types of TiO₂ crystal. They showed only diffraction patterns attributed to Ti metal (FIG. 12A). However, the UV-VIS absorption spectra for both titanium disks showed an absorption band at 300-350 nm (FIG. 12B). The absorption edge of the acid etched surface was in slightly longer wavelength regions than that of the machined surface.

X-ray photoelectron spectroscopy (XPS) spectra showed peaks of Ti2p, OIs and CIs for both titanium surfaces, but not other peaks, indicating the absence of impurity contamination other than these elements (FIG. 12C). The narrow spectrum of Ti2p revealed a clear 2p_{3/2} peak at approximately 458.5 eV with no shoulder peaks in the lower-energy regions (FIG. 12D). The 2p_{3/2} peak was slightly shifted to a higher degree for the acid-etched surface compared with the machined surface.

Chemical analysis of the acid-etched titanium surface was conducted to identify factors responsible for enhanced bioactivity. XPS spectra revealed that the C1s peak decreased with an increase of UV treatment time, whereas Ti2p and O1s peaks increased (FIG. 12E, F, G). Especially, a shoulder peak at about 288 eV ascribed to oxygen-containing hydrocarbons strongly adsorbed on TiO₂ surfaces disappeared. The atomic percentage of carbon continued to decrease up to 48 h of UV treatment from >50% to <20% (FIG. 12H). Least mean square approximation yielded a negative linear correlation between the atomic percentage of carbon and the amount of albumin adsorbed to the titanium surface, with a high coefficient of determination (R²=0.930); the less carbon on the titanium surface, the more albumin was adsorbed to the surface (FIG. 12I). The rate of osteoblast attachment yielded a different pattern of regression curve; it increased exponentially with the progressive removal of carbon (FIG. 12J). The contact angle did not significantly correlate with the rate of albumin adsorption or cell attachment (FIG. 12K, L).

17. Effective Use of a Combination of UVA and UVC to Produce Surface Electropositive Charge and Attract Cells.

As shown in FIG. 13, the use of both UVA and UVC light source increased most the number of cell attachment when compared to the use of UVA only or UVC only.

### REFERENCES

1. Campion, E. W., Jette, A. M., Cleary, P. D. & Harris, B. A. Hip fracture: a prospective study of hospital course, complications, and costs. J Gen Intern Med 2, 78-82 (1987).
2. Guccione, A. A., Fagerson, T. L. & Anderson, J. J. Regaining functional independence in the acute care setting following hip fracture. Phys Ther 76, 818-26 (1996).
3. Bhandari, M. et al. Predictors of in-hospital mortality following operative management of hip fractures. Int J Surg Investig 1, 319-26 (1999).
4. Melton, L. J., 3rd et al. Fractures attributable to osteoporosis: report from the National Osteoporosis Foundation. J Bone Miner Res 12, 16-23 (1997).
5. Espehaug, B., Fumes, O., Havelin, L. I., Engesaeter, L. B. & Vollset, S. E. The type of cement and failure of total hip replacements. J Bone Joint Surg Br 84, 832-8 (2002).
6. Hudson, J. I. et al. Eight-year outcome associated with clinical options in the management of femoral neck fractures. Clin Orthop Relat Res, 59-66 (1998).
7. Lu-Yao, G. L., Keller, R. B., Littenberg, B. & Wennberg, J. E. Outcomes after displaced fractures of the femoral neck. A meta-analysis of one hundred and six published reports. J Bone Joint Surg Am 76, 15-25 (1994).
8. Tidermark, J., Ponzer, S., Svensson, O., Soderqvist, A. & Tornkvist, H. Internal fixation compared with total hip replacement for displaced femoral neck fractures in the elderly. A randomised, controlled trial. J Bone Joint Surg Br 85, 380-8 (2003).
9. Ravikumar, K. J. & Marsh, G. Internal fixation versus hemiarthroplasty versus total hip arthroplasty for displaced subcapital fractures of femur—13 year results of a prospective randomised study. Injury 31, 793-7 (2000).
10. Tidermark, J., Zethraeus, N., Svensson, O., Tornkvist, H. & Ponzer, S. Quality of life related to fracture displacement among elderly patients with femoral neck fractures treated with internal fixation. 2002. J Orthop Trauma 17, S17-21 (2003).
11. LeGeros, R. Z. & Craig, R. G. Strategies to affect bone remodeling: osteointegration. J Bone Miner Res 8 Suppl 2, S583-96 (1993).
12. Puleo, D. A. & Nanci, A. Understanding and controlling the bone-implant interface. Biomaterials 20, 2311-21 (1999).
13. Pilliar, R. M. Cementless implant fixation-toward improved reliability. Orthop Clin North Am 36, 113-9 (2005).
14. Ozawa, S. et al. Ovariectomy hinders the early stage of bone-implant integration: histomorphometric, biomechanical, and molecular analyses. Bone 30, 137-43 (2002).
15. Yamazaki, M. et al. Bone reactions to titanium screw implants in ovariectomized animals. Oral Surg Oral Med Oral Pathol Oral Radiol Endod 87, 411-8 (1999).
16. Takeshita, F., Murai, K., Ayukawa, Y. & Suetsugu, T. Effects of aging on titanium implants inserted into the tibiae of female rats using light microscopy, SEM, and image processing. J Biomed Mater Res 34, 1-8 (1997).
17. Zhang, H., Lewis, C. G., Aronow, M. S. & Gronowicz, G. A. The effects of patient age on human osteoblasts' response to Ti-6Al-4V implants in vitro. J Orthop Res 22, 30-8 (2004).
18. Geertman, M. E. et al. Two-center clinical trial of implant-retained mandibular overdentures versus complete dentures-chewing ability. Community Dent Oral Epidemiol 24, 79-84 (1996).
19. Carlsson, G. E. & Lindquist, L. W. Ten-year longitudinal study of masticatory function in edentulous patients treated with fixed complete dentures on osseointegrated implants. Int J Prosthodont 7, 448-53 (1994).
20. Pera, P., Bassi, F., Schierano, G., Appendino, P. & Preti, G. Implant anchored complete mandibular denture: evaluation of masticatory efficiency, oral function and degree of satisfaction. J Oral Rehabil 25, 462-7 (1998).
21. van Kampen, F. M., van der Bilt, A., Cune, M. S., Fontijn-Tekamp, F. A. & Bosman, F. Masticatory function with implant-supported overdentures. J Dent Res 83, 708-11 (2004).
22. Nowjack-Raymer, R. E. & Sheiham, A. Association of edentulism and diet and nutrition in US adults. J Dent Res 82, 123-6 (2003).
23. Heydecke, G., McFarland, D. H., Feine, J. S. & Lund, J. P. Speech with maxillary implant prostheses: ratings of articulation. J Dent Res 83, 236-40 (2004).
24. Melas, F., Marcenes, W. & Wright, P. S. Oral health impact on daily performance in patients with implant-stabilized overdentures and patients with conventional complete dentures. Int J Oral Maxillofac Implants 16, 700-12 (2001).
25. van Steenberghe, D., Jacobs, R., Desnyder, M., Maffei, G. & Quirynen, M. The relative impact of local and endogenous patient-related factors on implant failure up to the abutment stage. Clin Oral Implants Res 13, 617-22 (2002).
26. Nevins, M. L., Karimbux, N. Y., Weber, H. P., Giannobile, W. V. & Fiorellini, J. P. Wound healing around endosseous implants in experimental diabetes. Int J Oral Maxillofac Implants 13, 620-9 (1998).
27. Doundoulakis, J. H., Eckert, S. E., Lindquist, C. C. & Jeffcoat, M. K. The implant-supported overdenture as an alternative to the complete mandibular denture. J Am Dent Assoc 134, 1455-8 (2003).
28. Annual industry report. US markets for dental implants: Executive summary. Implant Dent 12, 108-111 (2003).
29. Ellingsen, J. E. A study on the mechanism of protein adsorption to TiO2. Biomaterials 12, 593-6 (1991).
30. Klinger, A., Steinberg, D., Kohavi, D. & Sela, M. N. Mechanism of adsorption of human albumin to titanium in vitro. J Biomed Mater Res 36, 387-92 (1997).
31. Ogawa, T. et al. Biomechanical evaluation of osseous implants having different surface topographies in rats. J Dent Res 79, 1857-63 (2000).
32. Ray N F, Chan J K, Thamer M, Melton L J, 3rd. Medical expenditures for the treatment of osteoporotic fractures in the United States in 1995: report from the National Osteoporosis Foundation. J Bone Miner Res 1997; 12:24-35.
33. Jones L C, Hungerford D S. Cement disease. Clin Orthop Relat Res 1987: 192-206.
34. Espehaug B, Fumes O, Havelin L I, Engesaeter L B, Vollset S E. The type ofcement and failure of total hip replacements. J Bone Joint Surg Br 2002; 84:832-8.
35. Bhandari M, Koo H, Saunders L, Shaughnessy S G, Dunlop R B, Schemitsch E H. Predictors of in-hospital mortality following operative management of hip fractures. Int J Surg Investig 1999; 1:319-26.
36. Lamade W R, Friedl W, Schmid B, Meeder P J. Bone cement implantation syndrome. A prospective randomised trial for use of antihistamine blockade. Arch Orthop Trauma Surg 1995; 114:335-9.
37. Takeshita F, Murai K, Ayukawa Y, Suetsugu T. Effects of aging on titanium implants inserted into the tibiae of female rats using light microscopy, SEM, and image processing. J Biomed Mater Res 1997; 34:1-8.
38. Nowjack-Raymer R E, Sheiham A. Association of edentulism and diet and nutrition in US adults. J Dent Res 2003; 82:123-6.
39. Weinlaender M, Kenney E B, Lekovic V, Beumer J, 3rd, Moy P K, Lewis S. Histomorphometry of bone apposition around three types of endosseous dental implants. Int J Oral Maxillofac Implants 1992; 7:491-6.
40. Berglundh T, Abrahamsson I, Albouy J P, Lindhe J. Bone healing at implants with a fluoride-modified surface: an experimental study in dogs. Clin Oral Implants Res 2007; 18:147-52.
41. Ogawa T, Nishimura I. Different bone integration profiles of turned and acid-etched implants associated with modulated expression of extra cellular matrix genes. Int J Oral Maxillofac Implants 2003; 18:200-10.
42. De Maeztu M A, Braceras I, Alava J J, Gay-Escoda C. Improvement of osseointegration of titanium dental implant surfaces modified with CO ions: a comparative histomorphometric study in beagle dogs. Int J Oral Maxillofac Surg 2008; 37:441-7.
43. Moy P K, Medina D, Shetty V, Aghaloo T L. Dental implant failure rates and associated risk factors. Int J Oral Maxillofac Implants 2005; 20:569-77.
44. Esposito M, Hirsch J M, Lekholm U, Thomsen P. Failure patterns of four osseointegrated oral implant systems. J Mater Sci Mater Med 1997; 8:843-7.
45. Chuang S K, Wei U, Douglass C W, Dodson T B. Risk factors for dental implant failure: a strategy for the analysis of clustered failure-time observations. J Dent Res 2002; 81:572-7.
46. Wang R, Hashimoto K, Fujishima A. Light-induced amphiphilic surfaces. Nature 1997; 388:431-32.
47. Keleher J, Bashant J, Heldt N, Johnson L, Li YZ. Photo-catalytic preparation of silver-coated TiO2 particles for antibacterial applications. World J Microb Biot 2002; 18:133-39.
48. Nakashima T, Ohko Y, Kubota Y, Fujishima A. Photocatalytic decomposition of estrogens in aquatic environment by reciprocating immersion of TiO2-modified polytetrafluoroethylene mesh sheets. J Photoch Photobio A 2003; 160:115-20.
49. Saruwatari L, Aita H, Butz F, Nakamura H K, Ouyang J, Yang Y; et al. Osteoblasts generate harder, stiffer, and more delamination-resistant mineralized tissue on titanium than on polystyrene, associated with distinct tissue micro- and ultrastructure. J Bone Miner Res 2005; 20:2002-16.
50. Ogawa T, Ozawa S, Shih J H, Ryu K H, Sukotjo C, Yang J M, et al. Biomechanical evaluation of osseous implants having different surface topographies in rats. J Dent Res 2000; 79:1857-63.
51. Stein G S, Lian J B. Molecular mechanisms mediating proliferation/differentiation interrelationships during progressive development of the osteoblast phenotype. Endocr Rev 1993; 14:424-42.
52. Siddhanti S R, Quarles L D. Molecular to pharmacologic control of osteoblast proliferation and differentiation. J Cell Biochem 1994; 55:310-20.
53. Alborzi A, Mac K, Glackin C A, Murray S S, Zernik J H. Endochondral and intramembranous fetal bone development: osteoblastic cell proliferation, and expression of alkaline phosphatase, m-twist, and histone H4. J Craniofac Genet Dev Biol 1996; 16:94-106.
54. Owen T A, Aronow M, Shalhoub V, Barone L M, Wilming L, Tassinari M S, et al. Progressive development of the rat osteoblast phenotype invitro:reciprocal relationships in expression of genes associated with osteoblast proliferation and differentiation during formation of the bone extracellular matrix. J Cell Physiol 1990; 143:420-30.
55. Ogawa T, Sukotjo C, Nishimura I. Modulated bone matrix-related gene expression is associated with differences in interfacial strength of different implant surface roughness. J Prosthodont 2002; 11:241-7.
56. Takeuchi K, Saruwatari L, Nakamura H K, Yang J M, Ogawa T. Enhanced intrinsic biomechanical properties of osteoblastic mineralized tissue on roughened titanium surface. J Biomed Mater Res A 2005; 72A:296-305.
57. Bachle M, Kohal R J. A systematic review of the influence of different titanium surfaces on proliferation, differentiation and protein synthesis of osteoblast-like MG63 cells. Clin Oral Implants Res 2004; 15:683-92.
58. Zhao G, Schwartz Z, Wieland M, Rupp F, Geis-Gerstorfer J, Cochran D L, et al. High surface energy enhances cell response to titanium substrate microstructure. J Biomed Mater Res A 2005; 74:49-58.
59. Boyan B D, Bonewald L F, Paschalis E P, Lohmann C H, Rosser J, Cochran D L, et al. Osteoblast-mediated mineral deposition in culture is dependent on surface microtopography. Calcif Tissue Int 2002; 71:519-29.
60. Washburn N R, Yamada K M, Simon C G, Jr., Kennedy S B, Amis E J. High-throughput investigation of osteoblast response to polymer crystallinity:influence of nanometer-scale roughness on proliferation. Biomaterials 2004; 25:1215-24.
61. Zapata P, Su J, Garcia Al, Meredith J C. Quantitative high-throughput screening of osteoblast attachment, spreading, and proliferation on demixed polymer blend micropatterns. Biomacromolecules 2007; 8:1907-17.
62. Huang S, Chen C S, Ingber D E. Control of cyclin D1, p27(Kip1), and cell cycle progression in human capillary endothelial cells by cell shape and cytoskeletal tension. Mol Biol Cell 1998; 9:3179-93.
63. Sawase T, Jimbo R, Baba K, Shibata Y, Ikeda T, Atsuta M. Photo-induced hydrophilicity enhances initial cell behavior and early bone apposition. Clin Oral Implan Res 2008; 19:491-96.
64. Buser D, Broggini N, Wieland M, Schenk R K, Denzer Al, Cochran D L, et al. Enhanced bone apposition to a chemically modified SLA titanium surface. J Dent Res 2004; 83:529-33.
65. Massaro C, Rotolo P, De Riccardis F, Milella E, Napoli A, Wieland M, et al. Comparative investigation of the surface properties of commercial titanium dental implants. Part I: chemical composition. J Mater Sci Mater Med 2002; 13:535-48.
66. Takeuchi M, Sakamoto K, Martra G, Coluccia S, Anpo M. Mechanism of photoinduced superhydrophilicity on the TiO₂ photocatalyst surface. J Phys Chern B 2005; 109:15422-28.
67. Kikuchi L, Park J Y, Victor C, Davies J E. Platelet interactions with calcium-phosphate-coated surfaces. Biomaterials 2005; 26:5285-95.
68. Uetsuka H, Onishi H, Henderson M A, White J M. Photoinduced redox reaction coupled with limited electron mobility at metal oxide surface. J Phys Chern B 2004; 108:10621-24.
69. Henderson M A, White J M, Uetsuka H, Onishi H. Selectivity changes during organic photooxidation on TiO₂: Role of 0-2 pressure and organic coverage. J Catal 2006; 238:153-64.
70. Kojima N, Ozawa S, Miyata Y, Hasegawa H, Tanaka Y, Ogawa T. High-throughput gene expression analysis in bone healing around titanium implants by DNA microarray. Clin Oral Implants Res 2008; 19:173-81.
71. Brunski J B, Puleo D A, Nanci A. Biomaterials and biomechanics of oral and maxillofacial implants: current status and future developments. Intl Oral Maxillofac Implants 2000; 15:15-46.
72. Sykaras N, Iacopino A M, Marker V A, Triplett R G, Woody R D. Implant materials, designs, and surface topographies: their effect on osseointegration. A literature review. Int J Oral Maxillofac Implants 2000; 15:675-90.
73. Ogawa T, Nishimura I. Genes differentially expressed in titanium implant healing. J Dent Res 2006; 85:566-70.
74. Shalabi M M, Gortemaker A, Van't HofMA, Jansen J A, Creugers N H. Implant surface roughness and bone healing: a systematic review. J Dent Res 2006; 85:496-500.

1. A medical implant comprising a metallic surface, wherein the metallic surface comprises a metal oxide bearing a positive charge.

2. The medical implant of claim 1, wherein the metal is selected from the group consisting of titanium, platinum, tantalum, niobium, nickel, iron, chromium, cobalt, zirconium, aluminum, and palladium.

3. The medical implant of claim 1, wherein the metallic surface is substantially free of hydrocarbon.

4. The medical implant of claim 1, wherein the implant comprises a carrier material.

5. The medical implant of claim 1, wherein the implant surface comprises a metal oxide cation.

6. The medical implant of claim 5, wherein the metal oxide cation is a titanium oxide cation.

7. The medical implant of claim 1, wherein the implant surface is capable of attracting a protein or cell at an enhanced rate.

8. The medical implant of claim 7, wherein the cell is selected from the group consisting of human mesenchymal stem cell and osteoblastic cell and wherein the protein is selected from the group consisting of bovine serum albumin, fraction V, and bovine plasma fibronectin.

9. The method of claim 7, wherein the protein or cell attaches to the implant surface directly.

10. The medical implant of claim 1, wherein the implant surface is capable of enhancing tissue-implant integration and/or bone-implant integration.

11. The medical implant of claim 1, wherein the implant surface is capable of any of the following or combination thereof:
increasing adsorption of protein,
increasing osteoblast migration,
increasing attachment of osteoblasts,
increasing osteoblast spread,
increasing proliferation of osteoblast, and
increasing osteoblastic differentiation.

12. A method for functionalizing a medical implant, comprising
(1) providing a metallic implant surface, and
(2) treating the implant surface thereby causing the surface to be electro-positively charged.

13. The method of claim 12, wherein the treated surface attracts protein and/or cells at an enhanced rate.

14. The method of claim 12, wherein the surface is a titanium surface.

15. The method of claim 14, wherein the titanium surface comprises TiO₂.

16. The method of claim 12, wherein the treated surface is substantially free of hydrocarbon.

17. The method of claim 12, wherein the implant comprises a carrier material.

18. The method of claim 12, further comprising a step of processing the implant surface prior to the step of treating the implant surface, wherein the implant surface is processed by chemical etching, machining, or sandblasting.

19. The method of claim 12, wherein the implant surface is treated by ultraviolet (UV) light.

20. The method of claim 18, wherein the processed surface is treated by ultraviolet (UV) light.

21. The method of claim 19, wherein the UV light is of a wave-length selected from the group consisting of about 170 nm to about 270 nm and about 340 nm to about 380 nm.

22. The method of claim 19, wherein the surface is treated by a combination of a UV light of a wave-length of about 170 nm to about 270 nm and a UV light of wave-length of about 340 nm to about 380 nm.

23. The method of claim 19, wherein the treatment with UV light is over a period of time up to 48 hours.

24. The method of claim 22, wherein the treatment with UV light is over a period of time selected from the group consisting of 30 seconds, 1 minute, 5, minutes, 15 minutes, 30 minutes, 1 hour, 3 hours, 5 hours, 10 hours, 15 hours, 24 hour, 36 hours, and 48 hours.

25. The method of claim 12, wherein the treated surface comprises a metal oxide cation.

26. The method of claim 25, wherein the metal oxide cation is a titanium oxide cation.

27. The method of claim 13, wherein the cell is selected from the group consisting of human mesenchymal stem cell and osteoblastic cell and the protein is selected from the group consisting of bovine serum albumin, fraction V, and bovine plasma fibronectin.

28. The method of claim 13, wherein the protein or cell attaches to the treated implant surface directly.

29. The method of claim 12, wherein the treated implant surface has improved tissue-implant integration and/or bone-implant integration over the untreated implant surface.

30. The method of claim 12, wherein the treated implant surface has improved bone-forming capacity over the non-treated implant surface.

31. The method of claim 12, wherein the treated implant surface is capable of any of the following or combination thereof:
enhancing adsorption of protein over untreated implant surface,
increasing osteoblast migration,
increasing attachment of osteoblasts,
increasing osteoblast spread,
increasing proliferation of osteoblast, and
increasing osteoblastic differentiation.

32. A method of enhancing bone-implant integration or bone-formation comprising the method of claim 11.
